(19) United States
(12) Patent Application Publication
(10) Pub. No.: US 2012/0283313 A1
(43) Pub. Date: Nov. 8, 2012

(54) INHIBITION AND TREATMENT OF PROSTATE CANCER METASTASIS

(75) Inventors: Raymond C. Bergan, Chicago, IL (US); Karl A. Scheidt, Evanston, IL (US)

(73) Assignee: NORTHWESTERN UNIVERSITY, Evanston, IL (US)

(21) Appl. No.: 13/555,537

(22) Filed: Jul. 23, 2012

Related U.S. Application Data
(63) Continuation of application No. 12/250,351, filed on Oct. 13, 2008.
(60) Provisional application No. 60/979,712, filed on Oct. 12, 2007.

(51) Int. Cl. A61K 31/352; A61K 31/7105; C12N 5/09; C12N 9/99; A61P 35/04; C07D 311/36; G01N 33/573; C12Q 1/68; G01N 21/00; A61K 31/7088; C12Q 1/48

(52) U.S. Cl. 514/44 A; 514/456; 435/375; 435/184; 435/15; 549/403; 435/7.4; 435/6.13; 435/6.12; 435/6.1

(57) ABSTRACT

The present invention provides compounds and methods of inhibiting and treating metastatic prostate cancer. The compounds include MEK4 inhibitors. In another aspect the invention provides methods of identifying inhibitors of metastatic prostate cancer by screening for inhibitors of MEK4.

### CROSS-REFERENCE TO RELATED APPLICATION

The present application is a continuation of pending U.S. patent application Ser. No. 12/250,351, filed Oct. 13, 2008, which claims priority to expired U.S. Provisional Patent Application No. 60/979,712, filed Oct. 12, 2007, the entire disclosure of which are herein incorporated by reference in their entireties.

### STATEMENT REGARDING FEDERALLY SPONSORED RESEARCH OR DEVELOPMENT

This invention was made with government support under R21 CA099263 awarded by the National Institutes of Health. The government has certain rights in the invention.

### FIELD OF INVENTION

The present invention relates generally to the treatment of prostate cancer and in particular to the inhibition of prostate cancer metastasis. Thus, there are provided compounds and methods for treating prostate cancer metastasis by inhibition of MEK4 kinase.

### SUMMARY OF THE INVENTION

It has been discovered that the kinase MEK4 regulates prostate cancer cell invasion, a key step in the metastasis of prostate cancer. Inhibition of MEK4 blocks downstream activation of MMP-2 and cell invasion and increases cell adhesion. Accordingly, there are provided herein methods of inhibiting and treating prostate cancer metastasis with inhibitors of MEK4 activity. Furthermore, there are provided methods of screening for inhibitors of metastatic prostate cancer by testing compounds for inhibition of MEK4 activity. Also, compounds for use in methods described herein are disclosed, including anti-MEK4 antibodies, siRNA, genistein, and genistein analogs, e.g., isoflavones, isoflavanols, and isoflavanes.

### BRIEF DESCRIPTION OF THE DRAWINGS

FIG. 1 shows SDS-PAGE gels resulting from a Western blot analysis of MEK3, MEK4, and MEK 6 expression in six prostate cancer cell lines.

FIGS. 2A and 2B show prostate cancer cell invasiveness in the absence (2A) and presence (2B) of genistein.

FIGS. 3A, 3B, 3C and 3D show results of experiments assessing the pharmacological relevance of MEK4 as a target for prostate cancer therapy. FIG. 3A shows gels showing the expression of MEK4 in various prostate cancer cell lines in the presence and absence of siRNA specific for MEK4. FIGS. 3B and 3C are bar graphs that show the results of RT-PCR experiments detecting the level of transcript for MEK3 (3B) and MEK4 (3C) in various prostate cancer cell lines in the absence and presence of siRNA. FIG. 3D is a bar graph showing that knockdown of MEK4 with siRNA specific for MEK4 suppresses prostate cancer cell invasion and abrogates the effect of genistein.

FIGS. 4A and 4B show the effects of genistein on phosphorylation by or of MEK4. FIG. 4A is a gel showing that genistein inhibits phosphorylation of JNK3 by MEK4. FIG. 4B shows that in vivo, genistein does not block TGF-β stimulated phosphorylation of MEK4 itself.

FIG. 5A is a graph showing that genistein decreased metastasis but not tumor volume in a dose dependent fashion. FIG. 5B are graphs showing that genistein blocks activation of p38 MAP kinase in vivo by decreasing phosphorylation of p38 MAP kinase even while the total amount of p38 MAP kinase increased.

FIG. 6 is a graph showing that genes affected by genistein in man regulate cell motility in human prostate epithelial cells. Expression of HCF2 was decreased and expression of BASP1 was increased by genistein in man. In vitro, they lead to differences in invasion.

FIG. 7 shows the inhibition of MEK4 kinase activity by genistein in vitro.

FIG. 8 shows Matrix Metalloprotein-2 (MMP-2) transcript levels in normal prostate epithelial cells from human patients treated or untreated with genistein. Transcript levels were determined using quantitative RT-PCR.

FIG. 9 shows the results of cell invasion assays conducted with Compounds 1-16 using PC3M or PC3 cells according to the method of Example 2.

FIG. 10 shows the results of 3-day growth inhibition dimethylthiazol-diphenyltetrazolium bromide (MTT) assays conducted with Compounds 1-16. PC3-M human prostate cancer cells were treated with different concentrations of the indicated compound, and then MTT reduction as an indicator of cell viability was measured according to the method of Example 9.

### DEFINITIONS

As used herein, the term “MEK4 pathway protein” refers to proteins both upstream and downstream of MEK4, as well as MEK4 itself, that are related to cancer cell metastasis (e.g., in prostate cancer) and include, but is not limited to, the following proteins: MEK4 (MAP2K4; MKK4), p38 MAPK (MAPK14), MAPKAPK2 (MK2), HSP27 (HSB1), and MMP-2 (Matrix metallopeptidase 2).

As used herein, the term “MEK4 pathway nucleic acid” refers to nucleic acids that encode the MEK4 pathway proteins.

As used herein, the term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. In certain embodiments, the antibodies of the present invention are directed toward a MEK4 pathway protein (e.g., anti-MEK4, anti-p38 MAPK, anti-MEK4 pathway, anti-MAPKAPK2, anti-HSP27, and anti-MMP-2).

As used herein, the term “antibody fragments” refers to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments. The antibody fragments preferably retain at least part of the hinge and optionally the CHI region of an IgG heavy chain. In other preferred embodiments, the antibody fragments comprise at least a portion of the CH2 region or the entire CH2 region. In certain embodiments, the antibody fragments of the present invention are directed toward a MEK4 pathway protein.

As used herein, the term “functional fragment”, when used in reference to a monoclonal antibody, is intended to refer to a portion of the monoclonal antibody which still retains a functional activity. A functional activity can be, for example, antigen binding activity or specificity. Monoclonal antibody functional fragments include, for example, individual heavy or light or light chains and fragments thereof, such as VL, VH and Fd; monovalent fragments, such as Fv, Fab, and Fab′; bivalent fragments such as F(ab′)₂; single chain Fv (scFv); and Fc fragments. Such terms are described in, for example, Harlowe and Lane, Antibodies: A Laboratory Manual, Cold Spring Harbor Laboratory, New York (1989); Molec. Biology and

Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, Second Ed., Wiley-Liss, Inc., New York, N.Y. (1990), all of which are herein incorporated by reference. The term functional fragment is intended to include, for example, fragments produced by protease digestion or reduction of a monoclonal antibody and by recombinant DNA methods known to those skilled in the art.

As used herein, “humanized” forms of non-human (e.g., murine) antibodies are chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody may also comprise at least a portion of an immunoglobulin constant region (Fc), typically that of a human immunoglobulin. Examples of methods used to generate humanized antibodies are described in U.S. Pat. No. 5,225,539 to Winter et al. (herein incorporated by reference). In certain embodiments, the present invention employs humanized anti-MEK4 pathway protein antibodies.

As used herein, the term “hypervariable region” refers to the amino acid residues of an antibody which are responsible for antigen-binding. The hypervariable region comprises amino acid residues from a “complementarity determining region” or “CDR” (i.e. residues 24-34 (L1), 50-56 (L2) and 89-97 (L3) in the light chain variable domain and 31-35 (HI), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196: 901-917 (1987), hereby incorporated by reference in its entirety). “Framework” or “FR” residues are those variable domain residues other than the hypervariable region residues as defined herein.

As used herein, the term “siRNAs” refers to small interfering RNAs. In some embodiments, siRNAs comprise a duplex, or double-stranded region, which can be in the form of a hairpin of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants. In certain embodiments, the siRNAs target MEK4 pathway nucleic acid, such as the mRNA that encodes one of the MEK4 pathway proteins.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

### DETAILED DESCRIPTION

In one aspect, the present invention provides methods inhibiting and/or treating prostate cancer. The methods include administering a therapeutically effective amount of an isolated MEK4 inhibitor to a subject suffering from metastatic prostate cancer or at risk for metastatic prostate cancer. Isolated MEK4 inhibitors are MEK4 inhibitors that have either been purified in some way from a natural source or have been produced synthetically. MEK4 inhibitors suitable for use in the present methods include compounds which bind directly to MEK4 and, e.g., interfere with or inhibit MEK4 activity such as the phosphorylation of p38 MAP kinase. Other MEK4 inhibitors suitable for use in the present methods include compounds that reduce expression of MEK4. For example, MEK4 inhibitors that may be employed in the present methods include antibodies, isoflavones such as genistein, isoflavanols, isoflavanes, and molecules that interfere with MEK4 expression, such as siRNA and antisense oligonucleotides. In some embodiments the MEK4 inhibitor is not genistein.

The present invention provides therapeutic agents for treating prostate cancer. In particular embodiments, the therapeutic agents are small molecules, antibodies, or nucleic acid molecules (e.g., anti-sense or siRNA molecules) that inhibit a MEK4 pathway protein or MEK4 pathway nucleic acid. In particular embodiments, the MEK4 pathway protein or nucleic acid is MEK4. In other embodiments, the MEK4 protein is selected from the group consisting of: MEK4 (MAP2K4; MKK4), p38 MAPK (MAPK14), MAPKAPK2 (MK2), HSP27 (HSB1), and MMP-2 (Matrix metallopeptidase 2).

### 1. Small Molecules MEK4 Pathway Inhibitors

There are provided herein compounds for use in methods of treating or inhibiting metastasis of prostate cancer include compounds of Formula I. Formula I is:

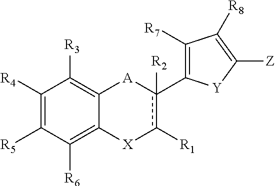

and stereoisomers, or pharmaceutically acceptable salts thereof, wherein,
A is O, C═O, CHOH, C═NR, or CH₂;
X is C═O, O or NH;
Y is O, NH, CR₉═CR₁₀, or CH═N;
Z is OH, OCH₃, halogen (F, Cl, Br, I), or may be H provided that one of R₇ or R₈ is OH or OCH₃;
the dashed line represents an optional double bond;
R is H or a substituted or unsubstituted alkyl group;
R₁ is selected from the group consisting of H and substituted or unsubstituted alkyl groups;
R₂ is selected from the group consisting of H, OH, F and Cl; or is absent when the optional double bond is present;
R₃, R₄, R₅, R₆, R₇, R₈, R₉ and R₁₀ are each independently selected from the group consisting of OH, F, Cl, Br, I, CN, NO₂, COOR, CONH₂, and substituted and unsubstituted alkyl and alkoxy groups; and
R is, at each occurrence, independently a substituted or unsubstituted alkyl or alkoxy group.

In some embodiments, the heterocyclic ring attached at the 2-position of the naphthalene scaffold (i.e., the ring attached at the carbon attached to the R₂ group in Formula 1, above) is instead provided at the 3 position (i.e., the carbon attached to the R₁ group in Formula 1, above). In some such embodiments, A is C═O and X is O.

In some embodiments, the compound of Formula I is not genistein (i.e., R₃ is not —OH, or R₄ is not —H, or R₅ is not —OH, or R₆ is not —H, or A is not C═O, or X is not O, or R₁ is not —H, or R₂ is not —H, or Y is not CR₉═CR₁₀ where R₉ and R₁₀ are —H, or Z is not —OH, or R₇ is not H, or R₈ is not H). For example, in some embodiments, the compound of Formula I lacks an —OH group at one or more of position R₃, R₅ or Z. For example, in some embodiments, the compound has R₃ and R₅ each independently is H, halogen, NO₂, COOR, CONH₂, or substituted and unsubstituted alkyl and alkoxy groups (e.g., R₃ and R₅ are each H; e.g., R₃, R₄, R₅, and R₆ are each H). Likewise, in some embodiments, Z is OCH₃, halogen, or H.

In some embodiments of compounds of Formula I, the double bond represented by the dashed line is present. Alternatively, in certain compounds of Formula I, the double bond represented by the dashed line is absent.

In compounds of Formula I, A can be C═O or CHOH. A may also be CH₂. In other embodiments, Y can be CR₉═CR₁₀. For example, Y can be CH═CH. In still other embodiments, Z can be OH. Compounds of Formula I also include compounds wherein A is C═O, the double bond represented by the dashed line is absent, and Y is CR₉═CR₁₀.

There are further provided herein compounds for use in methods of treating or inhibiting metastasis of prostate cancer include compounds of Formula II. Formula II is:

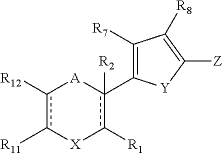

and stereoisomers, or pharmaceutically acceptable salts thereof, wherein,
A is O, C═O, CHOH, C═NR, or CH₂;
X is C═O, O or NH;
Y is O, NH, CR₉═CR₁₀, or CH═N;
Z is OH, OCH₃, halogen (F, Cl, Br, I), or may be H provided that one of R₇ or R₈ is OH or OCH₃;
the dashed lines represent optional double bonds;
R is H or a substituted or unsubstituted alkyl group;
R₁ is selected from the group consisting of H and substituted or unsubstituted alkyl groups;
R₆, R₇, R₈, R₉, R₁₀, R₁₁, and R₁₂ are each independently selected from the group consisting of OH, F, Cl, Br, I, CN, NO₂, COOR, CONH₂, and substituted and unsubstituted alkyl and alkoxy groups; and
R is, at each occurrence, independently a substituted or unsubstituted alkyl or alkoxy group.

In some embodiments, the heterocyclic ring attached at the 2-position of the naphthalene scaffold (i.e., the ring attached at the carbon attached to the R₂ group in Formula II, above) is instead provided at the 3 position (i.e., the carbon attached to the R₁ group in Formula 1, above). In some such embodiments, A is C═O and X is O.

In some embodiments of compounds of Formula II, one or both of the double bonds represented by the dashed lines are present. Alternatively, in certain compounds of Formula II, one or both of the double bonds represented by the dashed line are absent.

In compounds of Formula II, A can be C═O or CHOH. A may also be CH₂. In other embodiments, Y can be CR₉═CR₁₀. For example, Y can be CH=CH. In still other embodiments, Z can be OH. Compounds of Formula II also include compounds wherein A is C═O, the double bond represented by the dashed line is absent, and Y is CR₉═CR₁₀.

Existing therapies for the treatment of prostate cancer may be used in combination with the present methods. Thus methods of treating or inhibiting metastatic prostate cancer may further include administering the MEK4 inhibitor in conjunction with a second therapy for the treatment of prostate cancer. The second therapy may be another MEK4 inhibitor but typically is a different therapy. Suitable different therapies include one or more therapies selected from the group consisting of radiation treatment and prostatectomy. Another second therapy that may be used in is anti-androgen therapy. The anti-androgen therapy may include administering to the subject one or more agents selected from the group consisting of leuprolide and goserelin. Another second therapy that may be employed is chemotherapy such as administering one or more hormonal or chemotherapeutic agents that include but are not limited to ketoconazole, bicalutamide (Casodex), mitoxantrone (Novantrone), estramustine phosphate (Emcyt), etoposide (Vepsid), paclitaxel (Taxol), docetaxel (Taxotere), doxorubicin (Adriamycin), or vinblastine (Velban).

In another aspect, the invention provides methods of screening for compounds that inhibit prostate cancer metastasis comprising contacting MEK4 with one or more compounds in vitro and determining whether the compound inhibits MEK4. In some embodiments, the MEK4 is in a cell, e.g., in a cell culture system. In other embodiments, the MEK4 is an isolated enzyme. In some embodiments, the compounds are selected from the group consisting of isoflavones, isoflavanols, and isoflavanes.

The following abbreviations and terms are used throughout as defined below.

MEK4 is a kinase that phosphorylates p38 MAP kinase among other substrates and regulates prostate cancer cell motility and invasion.

PCa stands for prostate cancer.

SDS-PAGE stands for sodium dodecyl-sulfate polyacrylamide gel electrophoresis.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls(oxo); carboxyls; esters; urethanes; oximes; hydroxylamines; alkoxyamines; aralkoxyamines; thiols; sufides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitriles (i.e. CN); and the like.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched alkyl groups having from 1 to about 20 carbon atoms, and typically from 1 to 12 carbons or, in some embodiments, from 1 to 8, 1 to 6, or 1 to 4 carbon atoms. Alkyl groups further include cycloalkyl groups as defined below. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 10 or 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. In some embodiments, polycyclic cycloalkyl groups have three rings. Substituted cycloalkyl groups may be substituted one or more times with non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-, 2,5- or 2,6-disubstituted cyclohexyl groups, which may be substituted with substituents such as those listed above.

Bridged cycloalkyl groups are cycloalkyl groups in which two or more hydrogen atoms are replaced by an alkylene bridge, wherein the bridge can contain 2 to 6 carbon atoms if two hydrogen atoms are located on the same carbon atom, or 1 to 5 carbon atoms if the two hydrogen atoms are located on adjacent carbon atoms, or 2 to 4 carbon atoms if the two hydrogen atoms are located on carbon atoms separated by 1 or 2 carbon atoms. Bridged cycloalkyl groups can be bicyclic, such as, for example bicyclo[2.1.1]hexane, or tricyclic, such as, for example, adamantyl. Representative bridged cycloalkyl groups include bicyclo[2.1.1]hexyl, bicyclo[2.2.1]heptyl, bicyclo[3.2.1]octyl, bicyclo[2.2.2]octyl, bicyclo[3.2.2]nonyl, bicyclo[3.3.1]nonyl, bicyclo[3.3.2]decanyl, adamantyl, noradamantyl, bornyl, or norbornyl groups. Substituted bridged cycloalkyl groups may be substituted one or more times with non-hydrogen and non-carbon groups as defined above. Representative substituted bridged cycloalkyl groups may be mono-substituted or substituted more than once, such as, but not limited to, mono-, di- or tri-substituted adamantyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 20 carbon atoms, 4 to 16 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain and cycloalkyl groups as defined above, except that at least one double bond exists between two carbon atoms. Thus, alkenyl groups have from 2 to about 20 carbon atoms, and typically from 2 to 12 carbons or, in some embodiments, from 2 to 8, 2 to 6, or 2 to 4 carbon atoms. In some embodiments, alkenyl groups include cycloalkenyl groups having from 4 to 20 carbon atoms, 5 to 20 carbon atoms, 5 to 10 carbon atoms, or even 5, 6, 7 or 8 carbon atoms. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl, among others. Representative substituted alkenyl groups may be mono-substituted or substituted more than once, such as, but not limited to, mono-, di- or tri-substituted with substituents such as those listed above.

Cycloalkenylalkyl groups are alkyl groups as defined above in which a hydrogen or carbon bond of the alkyl group is replaced with a bond to a cycloalkenyl group as defined above. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8, 2 to 6, or 2 to 4 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups include nionocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6 to 10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 20 carbon atoms, 7 to 14 carbon atoms or 7 to 10 carbon atoms. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethylindanyl. Representative substituted aralkyl groups may be substituted one or more times with substituents such as those listed above.

Heterocyclyl groups are non-aromatic rings containing 3 or more ring members, of which one or more is a heteroatom such as, but not limited to, N, O, and S. In some embodiments, heterocyclyl groups include 3 to 20 ring members, whereas other such groups have 3 to 6, 3 to 10, 3 to 12, or 3 to 15 ring members. Heterocyclyl groups encompass partially unsaturated and saturated ring systems, such as, for example, imidazolinyl and imidazolidinyl groups. The phrase “heterocyclyl group” includes fused ring species including those comprising fused aromatic and non-aromatic groups, such as, for example, benzotriazolyl, 2,3dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups.” Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, pyrrolinyl, imidazolinyl, pyrazolinyl, thiazolinyl, piperidyl, piperazinyl, morpholinyl, thiomorpholinyl, tetrahydropyranyl, tetrahydrothiopyranyl, dihydropyridyl, dihydrodithiinyl, dihydrodithionyl, homopiperazinyl, quinuclidyl, indolinyl, indolizinyl. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups include at least one aromatic ring containing 5 or more ring members, of which one or more is a heteroatom such as N, O, and S. Heteroaryl groups include fused ring systems in which one or more rings are aryl or heterocyclyl such as indolyl, benzimidazolyl, and 5,6,7,8-tetrahydroquinolinyl. In some embodiments the heteroaryl group is a 5- or 6-member ring, a fused bicyclic ring having from 8-10 members, or a fused tricyclic ring having from 11 to 14 members. In other embodiments the heteroaryl group has 1, 2, 3, or 4 heteroatoms as ring members. Heteroaryl groups thus include, but are not limited to, groups such as pyrrolyl, pyrazolyl, imidazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl(pyrrolopyridyl), indazolyl, benzimidazolyl, imidazopyridyl(azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, 4-ethyl-morpholinyl, 4-propylmorpholinyl, furan-2-yl methyl, furan-3-yl methyl, pyridine-3-yl methyl, tetrahydrofuran-2-yl ethyl, and indol-2-yl propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl, or both the alkyl and heteroaryl portions of the group. Representative substituted heteroaralkyl groups may be substituted one or more times with substituents such as those listed above.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

Alkyl, alkenyl, and alkynyl groups maybe divalent as well as monovalent. The valency of an alkyl, alkenyl, or alkynyl group will be readily apparent from the context to those of skill in the art. For example, the alkyl group in an aralkyl group is divalent. In some embodiments, divalency is expressly indicated by appending the suffix “ene” or “ylene” to terms defined herein. Thus, for example, “alkylene” refers to divalent alkyl groups and alkenylene refers to divalent alkene groups.

The term “carboxylate” as used herein refers to a —COOH group.

The term “carboxylic ester” as used herein refers to —COOR³⁰ groups. R³⁰ is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., —C(O)NR³¹R³², and —NR³¹C(O)R³² groups, respectively. R³¹ and R³² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H).

Urethane groups include N- and O-urethane groups, i.e., —NR³³C(O)OR³⁴ and —OC(O)NR³³R³⁴ groups, respectively. R³³ and R³⁴ are independently hydrogen, or a substituted or unsubstituted alkyl, alkenyl, alkynyl, cycloalkyl, aryl, aralkyl, heterocyclylalkyl, or heterocyclyl group as defined herein.

The term “amine” (or “amino”) as used herein refers to —NHR³⁵ and —NR³⁶R³⁷ groups, wherein R³⁵, R³⁶ and R³⁷ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR³⁸R³⁹ and —NR³⁸SO₂R³⁹ groups, respectively. R³⁸ and R³⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include, but are not limited to, sulfamoyl groups (—SO₂NH₂).

The term “thiol” refers to —SH groups, while sulfides include —SR⁴⁰ groups, sulfoxides include —S(O)R⁴¹ groups, sulfones include —SO₂R⁴² groups, and sulfonyls include SO₂OR⁴³. R⁴⁰, R⁴¹, R⁴², and R⁴³ are each independently a substituted or unsubstituted alkyl, cycloalkyl, alkenyl, alkynyl, aryl aralkyl, heterocyclyl or heterocyclylalkyl group as defined herein.

The term “urea” refers to —NR⁴⁴—C(O)—NR⁴⁵R⁴⁶ groups. R⁴⁴, R⁴⁵, and R⁴⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁴⁷)NR⁴⁸R⁴⁹ and —NR⁴⁷C(NR⁴⁸)R⁴⁹, wherein R⁴⁷, R⁴⁸, and R⁴⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁵⁰C(NR⁵¹)NR⁵²R⁵³, wherein R⁵⁰, R⁵¹, R⁵² and R⁵³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁵⁴)═C(R⁵⁵)NR⁵⁶R⁵⁷ and —NR⁵⁴C(R⁵⁵)═C(R⁵⁶)R⁵⁷, wherein R⁵⁴, R⁵⁵, R⁵⁶ and R⁵⁷ are each independently hydrogen, a substituted or unsubstituted alkyl, cycloalkyl, alkenyl, alkynyl, aryl aralkyl, heterocyclyl or heterocyclylalkyl group as defined herein.

The term “imide” refers to —C(O)NR⁵⁸C(O)R⁵⁹, wherein R⁵⁸ and R⁵⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR⁶⁰(NR⁶¹) and —N(CR⁶⁰R⁶¹) groups, wherein R⁶⁰ and R⁶¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R⁶⁰ and R⁶¹ are not both simultaneously hydrogen.

Those of skill in the art will appreciate that compounds of the invention may exhibit the phenomena of tautomerism, conformational isomerism, geometric isomerism and/or optical isomerism. As the formula drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the compounds having one or more of the utilities described herein, as well as mixtures of these various different forms.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, triazoles may exhibit the following isomeric forms, which are referred to as tautomers of each other:

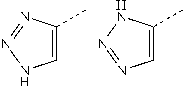

As readily understood by one skilled in the art, a wide variety of functional groups and other structures may exhibit tautomerism, and all tautomers of compounds as described herein are within the scope of the present invention.

Stereoisomers of compounds (also known as optical isomers) include all chiral, diastereomeric, and racemic forms of a structure, unless the specific stereochemistry is expressly indicated. Thus, compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

As used herein, a solvate is an aggregation of a molecule and one or more molecules of solvent. Some compounds have a tendency to associate with a fixed molar ratio of solvent molecules in the solid state. The solvent molecules may interact with the non-solvent molecule by dipole-dipole interactions, ion-dipole interactions, coordinate bonds, and the like. When the solvent is water, the solvate is referred to as a hydrate. Many organic solvents can also form solvates, including, e.g., ethers such as diethyl ether and tetrahydrofuran, alcohols such as methanol and ethanol, ketones such as acetone, DMF, DMSO and others. Solvates may be identified by various methods known in the art. For example, solvates in which the solvent molecules contain hydrogen may be observable by ¹H NMR. Additional methods useful in identifying solvates include thermogravimetric analysis, differential scanning calorimetry, X-ray analysis and elemental analysis. Solvates are readily formed simply by dissolving a compound in a solvent and removing the unassociated solvent by suitable techniques, e.g., evaporation, freeze drying or crystallization techniques. It is therefore well within the skill in the art to produce such solvates. Indeed, it is often the case that careful drying of a compound is necessary to remove the residual solvent that is part of a solvate. Compounds described herein may form solvates and all such solvates are within the scope of the invention.

Pharmaceutically acceptable salts of the invention compounds are considered within the scope of the present invention. When the compound of the invention has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., formic acid, acetic acid, fumaric acid, oxalic acid, tartaric acid, lactic acid, maleic acid, citric acid, succinic acid, malic acid, methanesulfonic acid, benzenesulfonic acid, and p-toluenesulfonic acid) or acidic amino acids (such as aspartic acid and glutamic acid). When the compound of the invention has an acidic group, such as for example, a carboxylic acid group, it can form salts with metals, such as alkali and earth alkali metals (e.g., Na⁺, Li⁺, K⁺, Ca₂⁺, Mg₂⁺, Zn₂⁺), ammonia, organic amines (e.g., trimethylamine, triethylamine, pyridine, picoline, ethanolamine, diethanolamine, triethanolamine), or basic amino acids (e.g., arginine, lysine and ornithine).

Compounds of Formula I may be synthesized by a variety of techniques known in the art. For example, Scheme 1 shows that aryl and heteroaryl boronic acids may be cross-coupled to 3-halo chromones (e.g., 3-bromochromone) via Suzuki coupling. Typical palladium catalysts, such as Pd(OAch)₂, and bases, such as potassium carbonate maybe used in this transformation. Additional methods for the synthesis of compounds of Formula I include one carbon homologations of deoxybenzoins (Wahala, et al., J. Chem. Soc.-Perkin Trans. 3005-3008 (1991); Balasubamanian, S. and Nair, M. G., Synth. Comm. 30:469-84 (2000); Chang, et al., J. Agric. Food Chem., 42:1869-71 (1994); hereby incorporated by reference in their entireties) and oxidative aryl isomerizations of chalcones induced by thallium(III) (McKillop, et al., Tet. Lett., 5281 (1970); Susse et al., Helv. Chim. Acta, 75:457-70 (199)) or hypervalent iodide (Prakash, et al., Synlett, 337-38 (1990); Kawamura et al., Synthesis, 2490-96 (2002)).

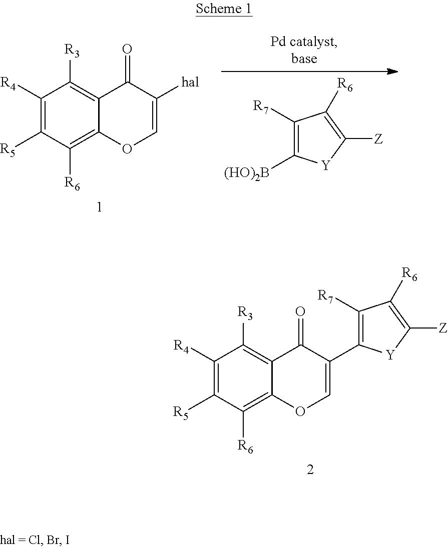

The resulting compound (2) may be further transformed by, e.g., conjugate addition of cuprates ((R₁)₂CuLi) to the unsaturated pyranone; imine formation at the ketone with armines (NHR), selective reduction of the ketone to either enantiomer by, e.g., diphenylpyrrolidinemethanol and 9-BBN (Kanth, J. V. B. and Brown, H. C. Tetrahedron, 58:1069-74 (2002)). Quinolone derivatives where X is N rather than O may be made by known methods similar to isoflavones. Traxler, et al., J. Med. Chem., 42:1018-26 (1999); Huang, et al., Biorg. Med. Chem., 6: 1657-62 (1998); Joseph, et al., Synlett, 1542-44 (2003).

### 2. Anti-MEK4 Pathway Antibodies

Described below are exemplary methods of generating anti-MEK4 pathway antibodies for use with the methods and systems of the present invention. The amino acid (and encoding nucleic acid) sequences of targeted human MEK4 pathway proteins, which are useful for generating antibodies, are as follows: MEK4 (NM_{—}003010), p38 MAPK (NM_{—}139013), MAPKAPK2 (NM_{—}004759), HSP27 (NM_{—}001540), and MMP-2 (NM_{—}004530).

(i) Polyclonal Antibodies

The present invention provides polyclonal antibodies directed toward MEK4 pathway proteins for use in the systems and methods of the present invention. Polyclonal antibodies are preferably raised in animals by multiple subcutaneous (sc) or intraperitoneal (ip) injections of the relevant antigen and an adjuvant. It may be useful to conjugate the MEK4 pathway protein or portion thereof to a protein that is immunogenic in the species to be immunized (e.g. keyhole limpet hemocyanin, serum albumin, bovine thyroglobulin, or soybean tyrpsin inhibitor) using a bifunctional or derivitizing agent (e.g. maleimidobenzoyl sulfosuccinimide ester for conjugation through cystein residues, N-hydroxysuccinimide for conjugation through lysine residues, glutaraldehyde, succinic anhydride, SOCl₂, or R1N═C═NR, where R and R1 are different alkyl groups.

Examples of a general immunization protocol for a rabbit and mouse are as follows.

Animals are immunized against a MEK4 pathway protein, MEK4 pathway protein-conjugates, or derivatives by combining, for example, 100 μg or 5 μg of the protein or conjugate (e.g. for a rabbit or mouse respectively) with 3 volumes of Freund's complete adjuvant and injecting the solution intradermally at multiple sites. One month later the animals are boosted with 1/5 or 1/10 the original amount of peptide or conjugate in Freund's complete adjuvant by subcutaneous injection at multiple sites. Seven to fourteen days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. In addition, aggregating agents such as alum are suitably used to enhance the immune response.

(ii) Monoclonal Antibodies

The present invention provides monoclonal antibodies that are specifically directed to MEK4 pathway proteins for use in the systems and methods of the present invention. Monoclonal antibodies may be made in a number of ways, including using the hybridoma method (e.g. as described by Kohler et al., Nature, 256: 495, 1975, herein incorporated by reference), or by recombinant DNA methods (e.g., U.S. Pat. No. 4,816,567, herein incorporated by reference).

In the hybridoma method, a mouse or other appropriate host animal, such as a hamster or macaque monkey, is immunized to elicit lymphocytes that produce or are capable of producing antibodies that will specifically bind to a MEK4 pathway protein. Alternatively, lymphocytes may be immunized in vitro. Lymphocytes then are fused with myeloma cells using a suitable fusing agent, such as polyethylene glycol, to form a hybridoma cell. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (e.g., Kozbor, J. Immunol., 133: 3001 (1984), herein incorporated by reference).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). After hybridoma cells are identified that produce antibodies of the desired specificity, affinity, and/or activity, the clones may be subcloned by limiting dilution procedures and grown by standard methods. Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies is described in more detail below.

In some embodiments, antibodies or antibody fragments are isolated from antibody phage libraries generated using the techniques described in, for example, McCafferty et al., Nature, 348: 552554 (1990). Clackson et al., Nature, 352:624-628 (1991) and Marks et al., J. Mol. Biol., 222: 581-597 (1991) describe the isolation of murine and human antibodies, respectively, using phage libraries. Subsequent publications describe the production of high affinity (nM range) human antibodies by chain shuffling (Marks et. al., BioTechnology, 10: 779-783 (1992)), as well as combinatorial infection and in vivo recombination as a strategy for constructing very large phage libraries (e.g., Waterhouse et al., Nuc. Acids. Res., 21: 2265-2266 (1993)). Thus, these techniques, and similar techniques, are viable alternatives to traditional monoclonal antibody hybridoma techniques for isolation of monoclonal antibodies.

Also, the DNA may be modified, for example, by substituting the coding sequence for human heavy-and light-chain constant domains in place of the homologous murine sequences (e.g., U.S. Pat. No. 4,816,567, and Morrison, et al., Proc. Nat. Acad. Sci USA, 81: 6851 (1984), both of which are hereby incorporated by reference), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized and Human Antibodies

The present invention provides humanized and human antibodies directed toward a MEK4 pathway protein for use in the methods and systems of the present invention. In certain embodiments, a humanized antibody comprises human antibody amino acid sequences together with amino acid residues that are not from a human antibody. In some embodiments, the human sequences in a humanized antibody comprise the framework regions (FRs) and the sequences or residues that are not from a human antibody comprise one or more complementarity-determining regions (CDRs).

The residues in a humanized antibody that are not from a human antibody may be residues or sequences imported from or derived from another species (including but not limited to mouse), or these sequences may be random amino acid sequences (e.g. generated from randomized nucleic acid sequences), which are inserted into the humanized antibody sequence. As noted above, the human amino acid sequences in a humanized antibody are preferably the framework regions, while the residues which are not from a human antibody (whether derived from another species or random amino acid sequences) preferably correspond to the CDRs. However, in some embodiments, one or more framework regions may contain one or more non-human amino acid residues. In cases of alterations or modifications (e.g. by introduction of a non-human residue) to an otherwise human framework, it is possible for the altered or modified framework region to be adjacent to a modified CDR from another species or a random CDR sequence, while in other embodiments, an altered framework region is not adjacent to an altered CDR sequence from another species or a random CDR sequence. In preferred embodiments, the framework sequences of a humanized antibody are entirely human (i.e. no framework changes are made to the human framework).

Non-human amino acid residues from another species, or a random sequence, are often referred to as “import” residues, which are typically taken from an “import” variable domain. Humanization can be essentially performed following the method of Winter and co-workers (e.g., Jones et al., Nature, 321: 522-525 (1986); Riechmann et al., Nature, 332: 323-327 (1988); Verhoeyen et al., Science, 239: 1534-1536 (1988), all of which are hereby incorporated by reference), by substituting rodent (or other mammal) CDRs or CDR sequences for the corresponding sequences of a human antibody. Also, antibodies wherein substantially less than an intact human variable domain has been substituted by the corresponding sequence from a non-human species may also be generated (e.g. U.S. Pat. No. 4,816,567, hereby incorporated by reference). In practice, humanized antibodies are typically human antibodies in which some CDR residues and possibly some FR residues are substituted by residues from analogous sites in rodent antibodies, or, as noted above, in which CDR sequences have been substituted by random sequences. By way of non-limiting example only, methods for conferring donor CDR binding affinity onto an antibody acceptor variable region framework are described in WO 01/27160 A1, herein incorporated by reference.

### 3. Nucleic Acid Based Agents

In certain embodiments, the present invention provides nucleic acid based agents (e.g., oligonucleotides) that target MEK4 pathway nucleic acids. In certain embodiments, the agents are siRNA molecules. In other embodiments, the agents are antisense molecules. The nucleic acid sequences of targeted human MEK4 pathway proteins, which are useful for generating antibodies, are as follows: MEK4 (NM_{—}003010), p38 MAPK (NM_{—}139013), MAPKAPK2 (NM_{—}004759), HSP27 (NM_{—}001540), and MMP-2 (NM_{—}004530). These sequences can be employed (e.g., using various software packages) to design RNAi and anti-sense sequences that target these genes or other genes of the MEK4 pathway.

i. RNA Interference (RNAi)

In some embodiments, RNAi is utilized to inhibit MEK4 pathway protein function by targeting MEK4 pathway nucleic acid. RNAi represents an evolutionary conserved cellular defense for controlling the expression of foreign genes in most eukaryotes, including humans. RNAi is typically triggered by double-stranded RNA (dsRNA) and causes sequence-specific mRNA degradation of single-stranded target RNAs homologous in response to dsRNA. The mediators of mRNA degradation are small interfering RNA duplexes (siRNAs), which are normally produced from long dsRNA by enzymatic cleavage in the cell. siRNAs are generally approximately twenty-one nucleotides in length (e.g. 21-23 nucleotides in length), and have a base-paired structure characterized by two nucleotide 3′-overhangs. Following the introduction of a small RNA, or RNAi, into the cell, it is believed the sequence is delivered to an enzyme complex called RISC (RNA-induced silencing complex). RISC recognizes the target and cleaves it with an endonuclease. It is noted that if larger RNA sequences are delivered to a cell, RNase III enzyme (Dicer) converts longer dsRNA into 21-23 nt ds siRNA fragments. In some embodiments, RNAi oligonucleotides are designed to target the junction region of fusion proteins.

Chemically synthesized siRNAs have become powerful reagents for genome-wide analysis of mammalian gene function in cultured somatic cells. Beyond their value for validation of gene function, siRNAs also hold great potential as gene-specific therapeutic agents (Tuschl and Borkhardt, Molecular Intervent. 2002; 2(3):158-67, herein incorporated by reference).

The transfection of siRNAs into animal cells results in the potent, long-lasting post-transcriptional silencing of specific genes (Caplen et al, Proc Natl Acad Sci U.S.A. 2001; 98: 9742-7; Elbashir et al., Nature. 2001; 411:494-8; Elbashir et al., Genes Dev. 2001; 15: 188-200; and Elbashir et al., EMBO J. 2001; 20: 6877-88, all of which are herein incorporated by reference). Methods and compositions for performing RNAi with siRNAs are described, for example, in U.S. Pat. No. 6,506,559, herein incorporated by reference.

siRNAs are extraordinarily effective at lowering the amounts of targeted RNA, and by extension proteins, frequently to undetectable levels. The silencing effect can last several months, and is extraordinarily specific, because one nucleotide mismatch between the target RNA and the central region of the siRNA is frequently sufficient to prevent silencing (Brummelkamp et al, Science 2002; 296:550-3; and Holen et al, Nucleic Acids Res. 2002; 30:1757-66, both of which are herein incorporated by reference).

An important factor in the design of siRNAs is the presence of accessible sites for siRNA binding. Bohula et al., (J. Biol. Chem., 2003; 278: 15991-15997; herein incorporated by reference) describe the use of a type of DNA array called a scanning array to find accessible sites in mRNAs for designing effective siRNAs. These arrays comprise oligonucleotides ranging in size from monomers to a certain maximum, usually synthesized using a physical barrier (mask) by stepwise addition of each base in the sequence. Thus, the arrays represent a full oligonucleotide complement of a region of the target gene. Hybridization of the target mRNA (e.g., MEK4 pathway nucleic acid) to these arrays provides an exhaustive accessibility profile of this region of the target mRNA. Such data are useful in the design of antisense oligonucleotides (ranging from 7 mers to 25 mers), where it is important to achieve a compromise between oligonucleotide length and binding affinity, to retain efficacy and target specificity (Sohail et al, Nucleic Acids Res., 2001; 29(10): 2041-2045). Additional methods and concerns for selecting siRNAs are described for example, in WO 05054270, WO05038054A1, WO03070966A2, J Mol Biol. 2005 May 13; 348(4):883-93, J Mol Biol. 2005 May 13; 348(4):871-81, and Nucleic Acids Res. 2003 Aug. 1; 31(15):4417-24, each of which is herein incorporated by reference in its entirety. In addition, software (e.g., the MWG online siMAX siRNA design tool) is commercially or publicly available for use in the selection of siRNAs.

ii. Antisense

In other embodiments, MEK4 pathway protein expression is modulated using antisense compounds that specifically hybridize with one or more MEK4 pathway nucleic acids encoding MEK4 pathway proteins. The specific hybridization of an oligomeric compound with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity that may be engaged in or facilitated by the RNA.

It is preferred to target specific nucleic acids for antisense. “Targeting” an antisense compound to a particular nucleic acid (e.g., a MEK4 pathway nucleic acid), in the context of the present invention, is a multistep process. The process usually begins with the identification of a nucleic acid sequence whose function is to be modulated. This may be, for example, a gene (or mRNA transcribed from the gene) in the MEK4 pathway whose expression is associated with a particular disorder or disease state, or a nucleic acid molecule from an infectious agent. In the present invention, the target is a MEK4 pathway nucleic acid molecule encoding a MEK4 peptide or other gene in the p38 MAPK pathway. The targeting process also includes determination of a site or sites within this gene for the antisense interaction to occur such that the desired effect, e.g., detection or modulation of expression of the protein, will result.

Within the context of the present invention, a preferred intragenic site is the region encompassing the translation initiation or termination codon of the open reading frame (ORF) of the gene. Since the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). Eukaryotic and prokaryotic genes may have two or more alternative start codons, any one of which may be preferentially utilized for translation initiation in a particular cell type or tissue, or under a particular set of conditions. In the context of the present invention, “start codon” and “translation initiation codon” refer to the codon or codons that are used in vivo to initiate translation of an mRNA molecule transcribed from a gene encoding a tumor antigen of the present invention, regardless of the sequence(s) of such codons.

Translation termination codon (or “stop codon”) of a gene may have one of three sequences (i.e., 5′-UAA, 5′-UAG and 5′-UGA; the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which refers to the region between the translation initiation codon and the translation termination codon, is also a region that may be targeted effectively. Other target regions include the 5′ untranslated region (5′ UTR), referring to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′ UTR), referring to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The cap region may also be a preferred target region.

Although some eukaryotic mRNA transcripts are directly translated, many contain one or more regions, known as “introns,” that are excised from a transcript before it is translated. The remaining (and therefore translated) regions are known as “exons” and are spliced together to form a continuous mRNA sequence. mRNA splice sites (i.e., intron-exon junctions) may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

In some embodiments, target sites for antisense inhibition are identified using commercially available software programs (e.g., Biognostik, Gottingen, Germany; SysArris Software, Bangalore, India; Antisense Research Group, University of Liverpool, Liverpool, England; GeneTrove, Carlsbad, Calif.). In other embodiments, target sites for antisense inhibition are identified using the accessible site method described in PCT Publ. No. WO0198537A2, herein incorporated by reference.

Once one or more target sites have been identified, oligonucleotides are chosen that are sufficiently complementary to the target (i.e., hybridize sufficiently well and with sufficient specificity) to give the desired effect. For example, in preferred embodiments of the present invention, antisense oligonucleotides are targeted to or near the start codon.

In the context of this invention, “hybridization,” with respect to antisense compositions and methods, means hydrogen bonding, which may be Watson-Crick, Hoogsteen or reversed Hoogsteen hydrogen bonding, between complementary nucleoside or nucleotide bases. For example, adenine and thymine are complementary nucleobases that pair through the formation of hydrogen bonds. It is understood that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired (i.e., under physiological conditions in the case of in vivo assays or therapeutic treatment, and in the case of in vitro assays, under conditions in which the assays are performed).

Antisense compounds are commonly used as research reagents and diagnostics. For example, antisense oligonucleotides, which are able to inhibit gene expression with specificity, can be used to elucidate the function of particular genes. Antisense compounds are also used, for example, to distinguish between functions of various members of a biological pathway.

The specificity and sensitivity of antisense is also applied for therapeutic uses. For example, antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides are useful therapeutic modalities that can be configured to be useful in treatment regimes for treatment of cells, tissues, and animals, especially humans.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e., from about 8 to about 30 linked bases), although both longer and shorter sequences may find use with the present invention. Particularly preferred antisense compounds are antisense oligonucleotides, even more preferably those comprising from about 12 to about 25 nucleobases.

Specific examples of preferred antisense compounds useful with the present invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In other preferred oligonucleotide mimetics, both the sugar and the internucleoside linkage (i.e., the backbone) of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science 254:1497 (1991).

Most preferred embodiments of the invention are oligonucleotides with phosphorothioate backbones and oligonucleosides with heteroatom backbones, and in particular —CH₂, —NH—O—CH₂—, —CH₂—N(CH₃)—O—CH₂— (known as a methylene(methylimino) or MMI backbone), —CH₂—O—N(CH₃)—CH₂—, —CH₂—N(CH₃)—N(CH₃)—CH₂—, and —O—N(CH₃)—CH₂—CH₂— (wherein the native phosphodiester backbone is represented as —O—P—O—CH₂—) of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta 78:486 [1995]) i.e., an alkoxyalkoxy group. A further preferred modification includes 2′-dimethylaminooxyethoxy (i.e., a O(CH₂)₂ON(CH₃)₂ group), also known as 2′-DMAOE, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other preferred modifications include 2′-methoxy(2′-O—CH₃), 2′-aminopropoxy(2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.° C. and are presently preferred base substitutions, even more particularly when combined with 2′-O-methoxyethyl sugar modifications.

Another modification of the oligonucleotides of the present invention involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, (e.g., hexyl-S-tritylthiol), a thiocholesterol, an aliphatic chain, (e.g., dodecandiol or undecyl residues), a phospholipid, (e.g., di-hexadecyl-rac-glycerol or triethylammonium 1,2-di-O-hexadecyl-rac-glycero-3-H-phosphonate), a polyamine or a polyethylene glycol chain or adamantane acetic acid, a palmityl moiety, or an octadecylamine or hexylamino-carbonyl-oxycholesterol moiety.

One skilled in the relevant art knows well how to generate oligonucleotides containing the above-described modifications. The present invention is not limited to the antisense oligonucleotides described above. Any suitable modification or substitution may be utilized.

It is not necessary for all positions in a given compound to be uniformly modified, and in fact more than one of the aforementioned modifications may be incorporated in a single compound or even at a single nucleoside within an oligonucleotide. The present invention also includes antisense compounds that are chimeric compounds. “Chimeric” antisense compounds or “chimeras,” in the context of the present invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNaseH is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the present invention may be formed as composite structures of two or more oligonucleotides, modified oligonucleotides, oligonucleosides and/or oligonucleotide mimetics as described above. The present invention also includes pharmaceutical compositions and formulations that include the antisense compounds of the present invention as described below.

In certain embodiments, the antisense sequences employed in the methods, compositions, and systems of the present invention are selected from the following:

(SEQ ID NO: 1)
5′-TTCCTCCTTTGTCTCCCAGC-3′;
(SEQ ID NO: 2)
5′-ATTCCTCCTTTGTCTCCCAG-3′;
(SEQ ID NO: 3)
5′-ATTCCTCCTTTGTCTCCCA-3′;
(SEQ ID NO: 4)
5′-GCCTCTTTATCACCTACCACA-3′;
(SEQ ID NO: 5)
5′-AAUUCCTCCTTTGTCUCCCA-3′;
(SEQ ID NO: 6)
5′-GUCUCTCTATGTGTGGGUUU-3′;
(SEQ ID NO: 7)
5′-UGUGUGTTCTCAGTCUCUCU-3′;
(SEQ ID NO: 8)
5′-CUCCUCGTCCAATTTCUCCA-3′;
and
(SEQ ID NO: 9)
5′-GGCUUGCTGTGGTCGAAGGC-3′.

Another use of oligonucleotides of the present invention involves direct contact between at least one oligonucleotide and at least one protein to form an aptameric interaction. Such an interaction may inhibit or otherwise affect the activity of a desired protein or proteins, such as MEK4 or MEK4 pathway members (see e.g., U.S. Pat. Nos. 5,998,596; 5, 270,163; 5,567,588; 5,595,877; 5,660,985; 5,696,249; 5,763,177; 5,817,785; 6,001,577; 6,184,364; 6,344,318; 6,376,190; 6,482,594; Bergan et al (1994) Nucleic Acids Res. 22:2150-54; Bergan et al (1995) Antisense Res. Dev. 5:33-8; Tuerk and Gold (1990) Science 249:505-10; Burke and Gold (1997) Nucleic Acids Res 25:2020-4; Brody et al (1999) Mol. Diagn. 4:381-88; Brody and Gold (2000) Rev. Mol. Biotechnol. 74:5-13; each herein incorporated by reference in their entireties).

### 4. Therapeutic Formulations and Uses

In some embodiments, the present invention provides therapeutic formulations comprising anti-MEK4 pathway agents (e.g., anti-MEK4 pathway antibodies, MEK4 pathway small molecules, and MEK4 pathway RNAi or antisense). It is not intended that the present invention be limited by the particular nature of the therapeutic composition. For example, such compositions can include an anti-MEK4 pathway agent, provided together with physiologically tolerable liquids, gels, solid carriers, diluents, adjuvants and excipients, and combinations thereof (See, e.g, Remington's Pharmaceutical Sciences 16th edition, Osol, A. Ed. (1980), herein incorporated by reference).

In addition, anti-MEK4 pathway agents may be used together with other therapeutic agents, including, but not limited to, salicylates, steroids, immunosuppressants, antibodies or antibiotics. Particular therapeutic agents which may be used with the anti-MEK4 pathway agents of the present invention include, but are not limited to, the following agents: azobenzene compounds (U.S. Pat. No. 4,312,806, incorporated herein by reference), benzyl-substituted rhodamine derivatives (U.S. Pat. No. 5,216,002, incorporated herein by reference), zinc L-carnosine salts (U.S. Pat. No. 5,238,931, incorporated herein by reference), 3-phenyl-5-carboxypyrazoles and isothiazoles (U.S. Pat. No. 5,294,630, incorporated herein by reference), IL-10 (U.S. Pat. No. 5,368,854, incorporated herein by reference), quinoline leukotriene synthesis inhibitors. (U.S. Pat. No. 5,391,555, incorporated herein by reference), 2′-halo-2′deoxy adenosine (U.S. Pat. No. 5,506,213, incorporated herein by reference), phenol and benzamide compounds (U.S. Pat. No. 5,552,439, incorporated herein by reference), tributyrin (U.S. Pat. No. 5,569,680, incorporated herein by reference), certain peptides (U.S. Pat. No. 5,756,449, incorporated herein by reference), omega-3 polyunsaturated acids (U.S. Pat. No. 5,792,795, incorporated herein by reference), VLA-4 blockers (U.S. Pat. No. 5,932,214, incorporated herein by reference), prednisolone metasulphobenzoate (U.S. Pat. No. 5,834,021, incorporated herein by reference), cytokine restraining agents (U.S. Pat. No. 5,888,969, incorporated herein by reference), p38 inhibitors (Herberich et al (2008) J. Med. Chem 10.1021/jm8005417; Cuenda et al (1995) FEBS Lett. 364:229-33; Jackson et al (1998) J. Pharmacol. Exper.Therapeutics 284:687-92; Young et al (1997) J Biol Chem 272:12116-21; Goedert et al (1997) EMBO J 16:3563-71; Buo et al (2005) Bioorg. Medicinal Chem. Lett. 16:64-8; WO/2007/126871; Xu et al (2008) FEBS Lett 8:1276-82; each incorporated herein by reference) and nicotine (U.S. Pat. No. 5,889,028, incorporated herein by reference).

Anti-MEK4 pathway agents may be used together with agents which reduce the viability or proliferative potential of a cell. Agents which reduce the viability or proliferative potential of a cell can function in a variety of ways including, for example, inhibiting DNA synthesis, inhibiting cell division, inducing apoptosis, or inducing non-apoptotic cell killing. Specific examples of cytotoxic and cytostatic agents, include but are not limited to, pokeweed antiviral protein, abrin, ricin, and each of their A chains, doxorubicin, cisplastin, iodine-131, yttrium-90, rhenium-188, bismuth-212, taxol, 5-fluorouracil VP-16, bleomycin, methotrexate, vindesine, adriamycin, vincristine, vinblastine, BCNU, mitomycin and cyclophosphamide and certain cytokines such as TNF-α and TNF-β. Thus, cytotoxic or cytostatic agents can include, for example, radionuclides, chemotherapeutic drugs, proteins, and lectins.

“Treating” within the context of the instant invention, means an alleviation, in whole or in part, of symptoms associated with a disorder or disease, or slowing, inhibiting or halting of further progression or worsening of those symptoms, or prevention or prophylaxis of the disease or disorder in a subject at risk for developing the disease or disorder. Thus, e.g., treating metastatic prostate cancer may include inhibiting or preventing the metastasis of the cancer, a reduction in the speed and/or number of the metastasis, a reduction in tumor volume of the metastasized prostate cancer, a complete or partial remission of the metastasized prostate cancer or any other therapeutic benefit. As used herein, a “therapeutically effective amount” of a compound of the invention refers to an amount of the compound that alleviates, in whole or in part, symptoms associated with a disorder or disease, or slows, inhibits or halts further progression or worsening of those symptoms, or prevents or provides prophylaxis for the disease or disorder in a subject at risk for developing the disease or disorder.

A subject is any animal that can benefit from the administration of a compound as described herein. In some embodiments, the subject is a mammal, for example, a human, a primate, a dog, a cat, a horse, a cow, a pig, a rodent, such as for example a rat or mouse. Typically, the subject is a human.

A therapeutically effective amount of a compound as described herein used in the present invention may vary depending upon the route of administration and dosage form. Effective amounts of invention compounds typically fall in the range of about 0.001 up to 100 mg/kg/day, and more typically in the range of about 0.05 up to 10 mg/kg/day. Typically, the compound or compounds used in the instant invention are selected to provide a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

Treatment may also include administering the compounds or pharmaceutical formulations of the present invention in combination with other therapies. Combinations of the invention may be administered simultaneously, separately or sequentially. For example, the compounds and pharmaceutical formulations of the present invention may be administered before, during, or after surgical procedure and/or radiation therapy. Alternatively, the compounds of the invention can also be administered in conjunction with other anticancer agents described herein. The specific amount of the additional active agent will depend on the specific agent used, the type of condition being treated or managed, the severity and stage of the condition, and the amount(s) of compounds and any optional additional active agents concurrently administered to the subject.

In certain embodiments, the present invention provides methods, systems, and compositions for both inhibiting a MEK4 pathway protein or nucleic acid and activating the endoglin-ALK2-Smad1 pathway so as to cause increased expression and/or activation of endoglin, ALK2, and/or Smad1. While the present invention is not limited to any particular mechanism, it is believed that inhibiting MEK4 signaling pathway and activating the endoglin-ALK2-Smad1 signaling pathway are both related to reducing cancer cell motility, particularly prostate cancer motility. As such, in certain embodiments, the MEK4 pathway inhibition described above is combined with compositions and methods for increasing the expression of endoglin, ALK2, and Smad1 in order to prevent cancer cell metastasis. In certain embodiments, small molecules are employed to increase the expression of proteins in the endoglin-ALK2-Smad1 pathway, such as genistein and genistein analogues. In other embodiments, expression vectors encoding endoglin, ALK2, or Smad1 are employed in gene therapy type methods to caused increased expression of the the genes encoding these proteins. The nucleic acid sequences encoding endoglin and Smad1 are are as follows: endoglin (NM_{—}000118), and Smad1 (NM_{—}001003688). These sequences can be employed to design appropriate expression vectors for causing increased expression of endoglin, ALK2, and Smad1.

In some embodiments of the invention, one or more compounds of the invention and an additional active agent are administered to a subject, more typically a human, in a sequence and within a time interval such that the compound can act together with the other agent to provide an enhanced benefit relative to the benefits obtained if they were administered otherwise. For example, the additional active agents can be co-administered by co-formulation, administered at the same time or administered sequentially in any order at different points in time; however, if not administered at the same time, they should be administered sufficiently close in time so as to provide the desired therapeutic or prophylactic effect. In some embodiments, the compound and the additional active agents exert their effects at times which overlap. Each additional active agent can be administered separately, in any appropriate form and by any suitable route. In other embodiments, the compound is administered before, concurrently or after administration of the additional active agents.

In various examples, the compound and the additional active agents are administered less than about 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In other examples, the compound and the additional active agents are administered concurrently. In yet other examples, the compound and the additional active agents are administered concurrently by co-formulation.

In other examples, the compound and the additional active agents are administered at about 2 to 4 days apart, at about 4 to 6 days apart, at about 1 week part, at about 1 to 2 weeks apart, or more than 2 weeks apart.

In certain examples, the inventive compound and optionally the additional active agents are cyclically administered to a subject. Cycling therapy involves the administration of a first agent for a period of time, followed by the administration of a second agent and/or third agent for a period of time and repeating this sequential administration. Cycling therapy can provide a variety of benefits, e.g., reduce the development of resistance to one or more of the therapies, avoid or reduce the side effects of one or more of the therapies, and/or improve the efficacy of the treatment.

In other examples, the inventive compound and optionally the additional active agent are administered in a cycle of less than about 3 weeks, about once every two weeks, about once every 10 days or about once every week. One cycle can comprise the administration of an inventive compound and optionally the second active agent by infusion over about 90 minutes every cycle, about 1 hour every cycle, about 45 minutes every cycle, about 30 minutes every cycle or about 15 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

Courses of treatment can be administered concurrently to a subject, i.e., individual doses of the additional active agents are administered separately yet within a time interval such that the inventive compound can work together with the additional active agents. For example, one component can be administered once per week in combination with the other components that can be administered once every two weeks or once every three weeks. In other words, the dosing regimens are carried out concurrently even if the therapeutics are not administered simultaneously or during the same day.

The additional active agents can act additively or, more typically, synergistically with the inventive compound. In one example, the inventive compound is administered concurrently with one or more second active agents in the same pharmaceutical composition. In another example, the inventive compound is administered concurrently with one or more second active agents in separate pharmaceutical compositions. In still another example, the inventive compound is administered prior to or subsequent to administration of a second active agent. The invention contemplates administration of an inventive compound and a second active agent by the same or different routes of administration, e.g., oral and parenteral. In certain embodiments, when the inventive compound is administered concurrently with a second active agent that potentially produces adverse side effects including, but not limited to, toxicity, the second active agent can advantageously be administered at a dose that falls below the threshold that the adverse side effect is elicited.

The instant invention also provides for pharmaceutical compositions and medicaments which may be prepared by combining one or more compounds described herein, pharmaceutically acceptable salts thereof, stereoisomers thereof, tautomers thereof, or solvates thereof, with pharmaceutically acceptable carriers, excipients, binders, diluents or the like to inhibit or treat primary and/or metastatic prostate cancers. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, or via implanted reservoir. Parenteral or systemic administration includes, but is not limited to, subcutaneous, intravenous, intraperitoneal, and intramuscular injections. The following dosage forms are given by way of example and should not be construed as limiting the instant invention.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or antioxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or phannaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

Compounds of the invention may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. Formulations for inhalation administration contain as excipients, for example, lactose, polyoxyethylene-9-lauryl ether, glycocholate and deoxycholate. Aqueous and nonaqueous aerosols are typically used for delivery of inventive compounds by inhalation.

Ordinarily, an aqueous aerosol is made by formulating an aqueous solution or suspension of the compound together with conventional phannaceutically acceptable carriers and stabilizers. The carriers and stabilizers vary with the requirements of the particular compound, but typically include nonionic surfactants (TWEENs, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions. A nonaqueous suspension (e.g., in a fluorocarbon propellant) can also be used to deliver compounds of the invention.

Aerosols containing compounds for use according to the present invention are conveniently delivered using an inhaler, atomizer, pressurized pack or a nebulizer and a suitable propellant, e.g., without limitation, pressurized dichlorodifluoromethane, trichlorofluoromethane, dichlorotetrafluoroethane, nitrogen, air, or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch. Delivery of aerosols of the present invention using sonic nebulizers is advantageous because nebulizers minimize exposure of the agent to shear, which can result in degradation of the compound.

For nasal administration, the pharmaceutical formulations and medicaments may be a spray, nasal drops or aerosol containing an appropriate solvent(s) and optionally other compounds such as, but not limited to, stabilizers, antimicrobial agents, antioxidants, pH modifiers, surfactants, bioavailability modifiers and combinations of these. For administration in the form of nasal drops, the compounds maybe formulated in oily solutions or as a gel. For administration of nasal aerosol, any suitable propellant may be used including compressed air, nitrogen, carbon dioxide, or a hydrocarbon based low boiling solvent.

Dosage forms for the topical (including buccal and sublingual) or transdermal administration of compounds of the invention include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Transdermal patches have the added advantage of providing controlled delivery of a compound of the invention to the body. Such dosage forms can be made by dissolving or dispersing the agent in the proper medium. Absorption enhancers can also be used to increase the flux of the inventive compound across the skin. The rate of such flux can be controlled by either providing a rate controlling membrane or dispersing the compound in a polymer matrix or gel.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

The formulations of the invention may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### EXAMPLES

### Example 1

MEK expression was examined in six human prostate cancer (PCa) cell lines: PC3 and PC3-M metastatic PCa cells, 1532CPTX and 1542 CPTX immortalized localized PCa cells, and 1532 NPTX and 1542 NPTX immortalized normal epithelial cells. The last four cell lines are primary cells, are HPV transformed, and thus represent early stages of prostate carcinogenesis. They provide representative members of the metastatic phenotype, as well as members of early state phenotypes. (Liu et. al., “Prostate cancer chemoprevention agents exhibit selective activity against early stage prostate cancer cells,” Prostate Cancer Prostatic Dis. 2001, 4: 81-91, herein incorporated by reference in its entirety). All six cell lines also secrete as well as respond to TGFβ, a regulator of cell motility that plays a role in PCa cell invasiveness.

A Western blot analysis of the six cell lines was performed. MEK Western blot analyses used identical amounts of protein and were exposed at the same time, allowing for comparison.

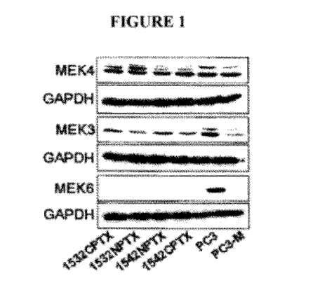

Results are shown in FIG. 1. MEK4 expression is high in all six cell lines, while MEK3 and MEK6 expression is low and variable.

### Example 2

The invasiveness of the PCa cell lines used in Example 1 was assessed in the absence and in the presence of genistein. Assays were conducted using methods as described in Craft et al. (2008, Mol. Pharmocol., 73(1):235-242; herein incorporated by reference in its entirety).

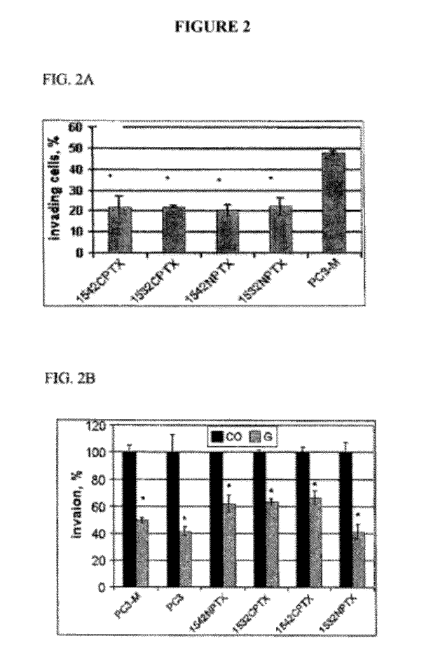

The results of this assay are presented in FIGS. 2A and 2B. FIG. 2A shows that early stage PCa cells are less invasive than metastatic PC3-M cells. FIG. 2B shows that genistein inhibits invasion of both early and late stage PCa cells.

### Example 3

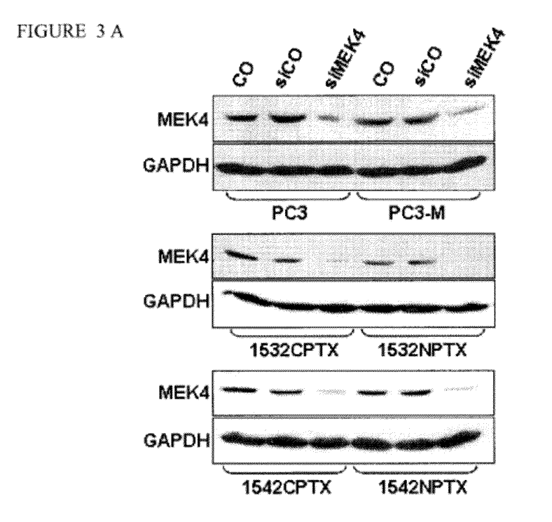

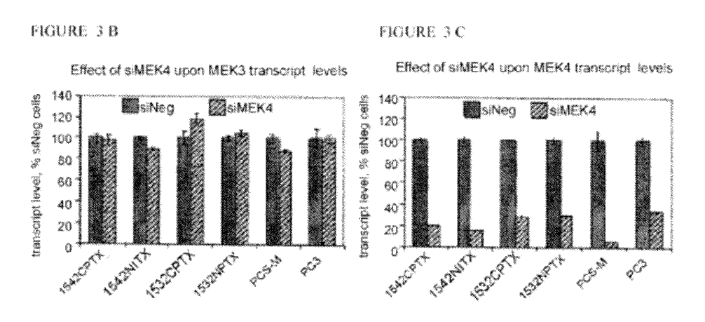

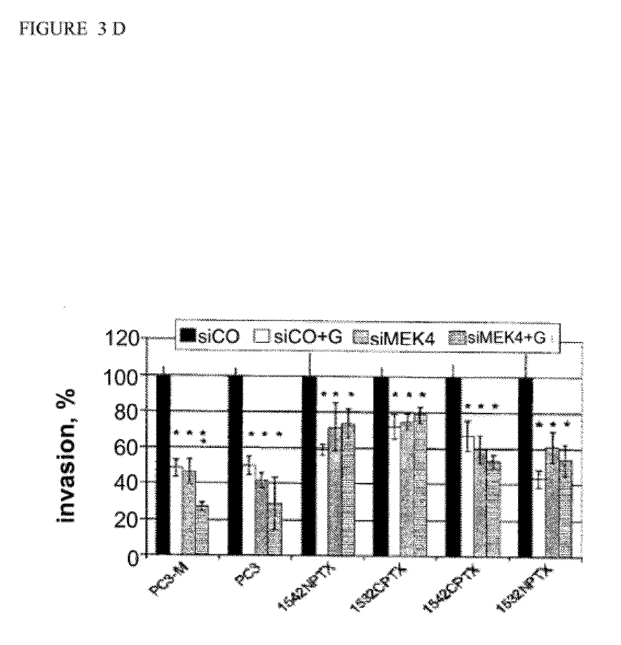

To show that MEK4 is a pharmacologically relevant target of genistein, a MEK4 knockdown experiment with MEK4 siRNA (siMEK4) was conducted using standard techniques. Results shown in FIG. 3A demonstrate that siMEK4 suppresses expression of MEK4 protein relative to non-targeting siRNA and untransfected controls. As a further control, the same human PCa cell lines were transfected with siMEK4 or non-targeting siNeg, and MEK3 and MEK4 transcript levels were measured using quantitative RT/PCR (values normalized to GAPDH). FIG. 3B shows that siMEK4 had no effect on MEK3 transcript levels, while FIG. 3C shows that siMEK4 significantly reduced MEK4 transcript levels in the same cells. Thus, the results show that siMEK4 is specific for MEK4 and does not suppress the homologous MEK3. (MEK6 is not expressed in most of these cell lines and was not examined).

The invasiveness of PCa cells in the presence genistein and siMEK4 was examined. FIG. 3D shows that when MEK4 expression was suppressed by siMEK4, the effect of genistein was abrogated.

### Example 4

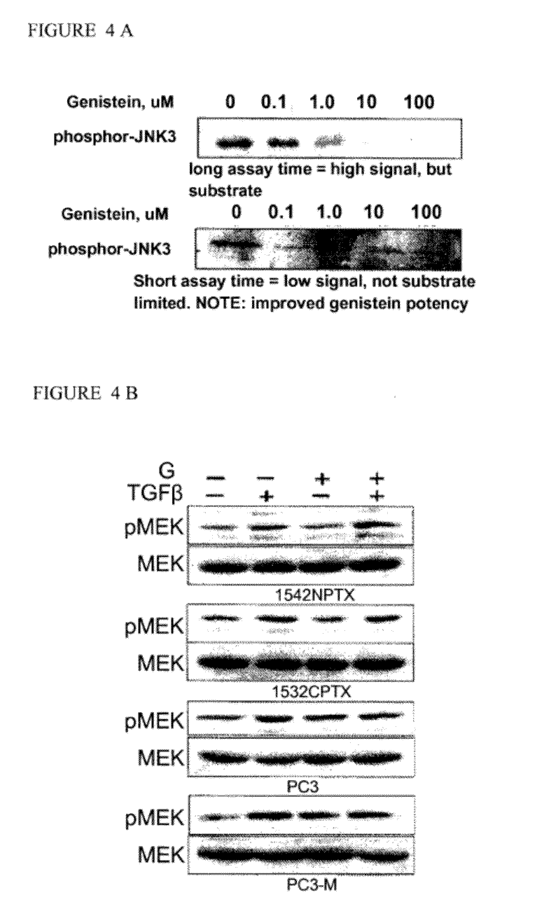

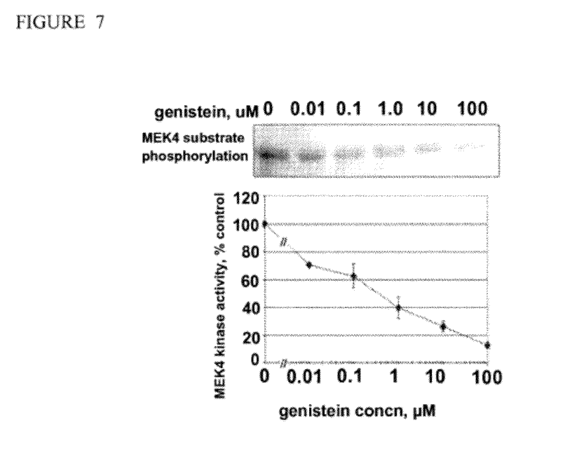

Phosphorylation by MEK4 (FIG. 4A) and phosphorylation of MEK4 (FIG. 7) was assayed. The Upstate Biotechnology MEK4 assay system was used to measure inhibition of MEK4 activity. Phosphorylation of MEK4 in vivo was assayed using standard techniques. The IC₅₀ of genistein with regard to inhibition of phosphorylation under these conditions is estimated to be less than 0.1 μM.

FIG. 4A shows that genistein inhibits phosphorylation of JNK3 by MEK4 in vitro. FIG. 4B demonstrates that TGFβ increases MEK4 phosphorylation in vivo but that genistein does not block such phosphorylation.

### Example 5

The ability of genistein to inhibit human PCa metastasis was examined using the following procedure. Inbred four-week old male athymic mice (Charles River Laboratories), were fed soy-free Harlan Teklad 20168 chow containing 0, 100, or 250 mg genistein/kg chow, beginning one week prior to implantation of 10⁶ human PC3-M PCa cells into the dorsal lobe of the prostate. Mice were necropsied four weeks later. There were 5 mice in each of the three dosing cohorts per experiment, X2 separate experiments which gave essentially identical outcomes, for a total of 30 mice. The resultant blood concentrations of total genistein were measured as described (Takimoto, et al. “Phase I pharmacokinetic and pharmacodynamic analysis of unconjugated soy isoflavones administered to individuals with cancer,” Cancer Epidemiol. Biomarkers Prevo 12:1213-21 (2003); herein incorporated by reference in its entirety), and were below the limits of quantitation (for controls), 290±72 nM (100 mg cohort), and 1307±507 nM (250 mg cohort). Knowing that free genistein is about a tenth of the total, gives estimated free concentrations of 29 nM and 131 nM. Such concentrations approximate the mean free concentrations reported in the blood of soy consuming Japanese men (Adlercreutz, et al., “Plasma concentrations of phyto-oestrogens in Japanese men,” Lancet, 342:1209-10 (1993); herein incorporated by reference in its entirety) and in men after prospective dosing with supradietary amounts of genistein (Takimoto, et al., Cancer Epidemiol. Biomarkers Prevo 12:1213-21 (2003); herein incorporated by reference in its entirety).

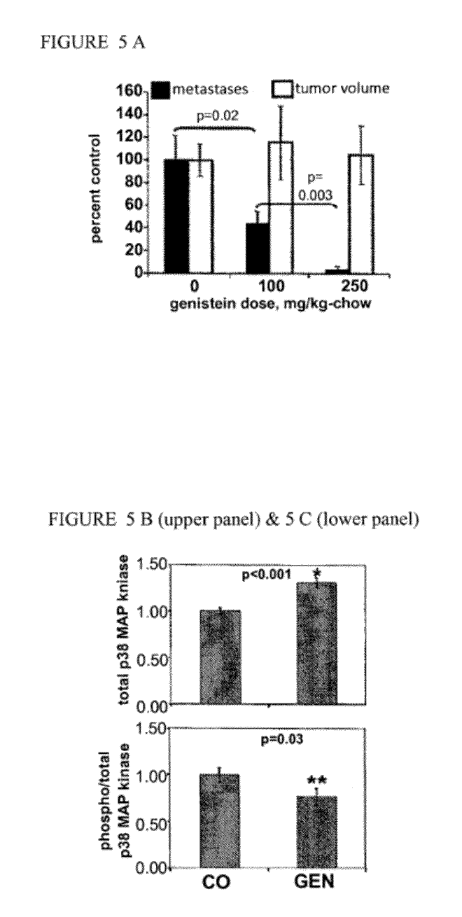

As shown in FIG. 5A, genistein decreased metastasis but not tumor volume in a dose dependent fashion. There was no difference in the weight of mice between cohorts. Western blot analysis of fresh frozen primary tumor tissue revealed that genistein increased the level of total p38 MAP kinase protein, but decreased its phosphorylation, as shown in FIG. 5B. The increase in “promotility” proteins likely represents a compensatory response by inherently metastatic cells to therapy which inhibits their motility. These findings demonstrate that genistein inhibits human PCa metastasis in a dose-responsive fashion in vivo at concentrations attained in the blood of men. Importantly, genistein still blocked the activation of p38 MAP kinase, even in the face of up-regulation. Finally, both in vivo and in vitro studies support dose escalation as a viable strategy for inhibiting metastasis of human prostate cancer.

### Example 6

Change in cell morphology is a generally recognized measure of change in cell adhesion. Compounds which increase cell adhesion of prostate cancer cells in vivo may inhibit prostate cancer metastasis. The effect of genistein on cell detachment was investigated in vivo. Quantitative image analysis according to established methods was used to measure in vivo changes in nuclear morphology in the prostate. (Bartels, et al., Prostate, 48:144-55, (2001); Boone, et al., Urology, 57:129-31 (2001); Bartels, et al., Anal. Quant. Cytol. Histol. 20:397-406 (1998); Bartels et al., Anal. Quant. Cytol. Histol. 20:389-96; Veltri, et al., J. Cell Biochem. Suppl., 151-57 (2000); herein incorporated by reference in their entireties).

Mouse: From the mouse experiment of Example 5, primary (prostate gland) and metastatic (local lymph nodes) tissue was Feulgen-stained, and the nuclear morphology of PC3M cells was quantitated on a ChromaVision ACIS® II Image Analysis System. Over 500 cells for each tissue type from mice treated with 250 mg genistein (N=5) or controls (N=5) were scored in a blinded fashion. Genistein was thereby shown to increase nuclear flattening in vivo. Specifically, for lymph node: cell area increased by 19.5±2.1%, cell length by 9.1±1.1%, and cell width by 9.5±1.1% (p:S 0.01 for all). For primary tumor: cell length increased by 3.0±1.1% (p:S 0.05). Thus, genistein induces nuclear flattening in vivo, a marker indicative of decreased cell detachment.

Humans: Genistein was administered to men with prostate cancer in a phase 1 pharmacokinetic/pharmacodynamic study of genistein, (Takimoto, et al. Cancer Epidemiol. Biomarkers Prev., 12:1213-21 (2003) herein incorporated by reference in its entirety), and a phase 2 study biomarker based study.

Phase 1 study: Doses from 2 to 8 mg genistein/kg (i.e., 2-32× dietary doses; considering that estimates of average daily genistein consumption by soy consumers ranges from 0.3 to 1 mg/kg) were administered to men with prostate cancer. Key findings include that: genistein was well tolerated, peak concentrations of total and free genistein ranged from 4.3-16.3 nM and 66-170 nM, respectively (i.e., >90% of blood genistein was conjugated, and thus inactive), halflife was 15-22 hrs, and clearance was not altered by body mass. These findings demonstrate that administration of genistein to a cohort of older men gives blood concentrations of free genistein associated with anti-metastatic efficacy in preclinical models.

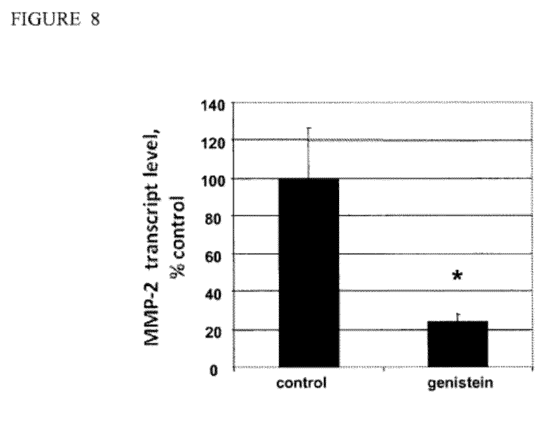

Phase 2 study: A Phase 2 trial of genistein in men with localized prostate cancer was conducted. Men were randomized (1:1) to treatment, or not, with 2 mg genistein/kg/day prior to radical prostatectomy (i.e., ˜2-8× average dietary dose). Genistein was given as a single pill/day for 1 month prior to surgery, using the same formulation used in the Phase 1 study (90% genistein; ˜0% daidzein, and thus no equol produced in people; Takimoto, C. H., et al, Cancer Epidemiol Biomarkers Prev, 2003, 12(11 Pt 1): p. 1213-21; herein incorporated by reference in its entirety). The mean±SEM trough concentration of free genistein for genistein treated and control subjects in the Phase 2 study was 26.6±6.6 nM and below detection, respectively. Of 38 subjects completing the study, MMP-2 expression was analyzed in tissue from 12 genistein-treated subjects and 12 controls. Patient characteristics did not differ between treatment and control cohorts (Table 1). MMP-2 expression was measured by removing normal prostate epithelial cells from intact fresh frozen prostate tissue by laser capture microdissection (LCM), isolating RNA, treating with DNase, assessing RNA quality by capillary electrophoresis, and measuring MMP-2 transcript levels by qRT/PCR (normalizing to GAPDH), using exon spanning primers. Genistein decreased MMP-2 to 24±4.1% of controls (mean±SEM; 2 sided t test p value=0.045) (FIG. 8).

TABLE 1
Study subject characteristics

| | treat- | | con- | | p |
|---|---|---|---|---|---|
| | ment | | trol | | value* |
| subjects, number | 12 | | 12 | | |
| age, mean (range) | 57 | (44-67) | 58 | (48-73) | NS |
| race | | | | | |
| caucasian, number (%) | 9 | 75 | 9 | 75 | NS |
| African American, number | 2 | 17 | 2 | 17 | NS |
| (%) | | | | | |
| other, number (%) | 1 | 8 | 1 | 8 | NS |
| clinical stage | | | | | |
| T1, number (%) | 7 | 58 | 6 | 50 | NS |
| T2, number (%) | 4 | 33 | 4 | 33 | NS |
| unknown, number (%) | 1 | 8 | 2 | 17 | NS |
| PSA, mean (SEM) | 6 | 0.57 | 6 | 0.61 | NS |
| Gleason score | | | | | |
| 6, number (%) | 7 | 58 | 7 | 58 | NS |
| 7, number (%) | 5 | 42 | 5 | 42 | NS |
| pre-surgery treatment time, | 4 | 0.6 | N/A** | N/A | |
| mean wks (SEM) | | | | | |

| | | | |
|---|---|---|---|
| serious adverse events¶, | 0 | 0 | NS |
| number | | | |

*2 sided t test p values > 0.05 are considered not significant (NS) for differences between treatment and control cohorts
**N/A not applicable
¶grade >/= 2 clinical toxicity according to the NCI Common Toxicity Criteria v2.0

The effect of genistein upon the nuclear morphology of prostate epithelial cells was investigated similarly as for the mouse cells. Genistein induces flattening of “normal” prostate epithelial cells in man. Though morphologically “normal,” these cells are present within organs with PCa, have pre-cancer molecular changes, and represent an appropriate target cell type for therapy that inhibits a process associated with PCa progression, in this case, development of the metastatic phenotype. Quantitative image analysis of nuclear morphology of >1000 cells per treatment cohort were scored from 6 genistein treated men, and 5 controls. Genistein increased: length by 1.5±0.7% (p<0.01), width by 2.7±0.7% (p<0.01), and area by 2.0±1.0% (this was only a trend; p=0.15). These studies indicate that genistein is inhibiting the detachment of prostate epithelial cells in man. These findings are consistent with its effects in vitro and in mice. They demonstrate that genistein is therapeutically inhibiting in a man a cellular process, in a relevant target cell type, linked to the development of metastasis.

The effects of genistein on genes which regulate cell motility were investigated using known techniques in gene array technology. (Jovanovic, et al., Am. J. Pharmacogenomics, 1: 145-52 (2001); Jovanovic, et al., Cancer Treat. Res., 113:91-111 (2002); Ding, et al., Prostate Cancer Prostatic Dis., 9:379-91 (2006); herein incorporated by reference in its entirety). In particular, methodology was employed wherein prostate epithelial cells are selectively removed from human prostate tissue by laser capture microdissection (LCM), the resultant RNA linear amplified, and custom manufactured 12K gene arrays are probed. Ding, et al., Id. This methodology was applied to 14 control and 10 genistein-treated subjects on the phase 2 trial, using statistical methods previously described (Jovanovic, et al., J. Probability, Statistics, and Quant. Management, 1:51-60 (2004), Ding, et al., Id.; herein incorporated by reference in their entireties), 6 genes were found to be altered by genistein in a statistically significant fashion (see Table 2). Of these 6 genes, 3 (or 1/2) have direct links to cell motility in other cell types. Specifically, heparin cofactor II (HCF2) induces formation of filamentous-actin and promotes cell migration (Hoffman, et al., Biochim. Biophys. Acta, 1095:78-82 (1995)), brain acid soluble protein 1 (BASP1) binds to the actin cytoskeleton and regulates its dynamic function (Frey et al., J. Cell Biol., 149:1443-54 (2000); Laux et al., J. Cell Biol., 149:1455-72 (2000); Wiederkehr et al., Exp. Cell Res., 236:103-16 (1997); herein incorporated by reference in its entirety), and MALATI (metastasis associated in lung adenocarcinoma transcript) is uniquely over expressed in metastatic lung cancer (Ji, et al., Oncogene 22:8031-41 (2003); herein incorporated by reference in its entirety). Further studies therefore focused upon the 3 motility-associated genes.

TABLE 2
Expression Levels of Genistein Responsive Genes

| | | | | qRT/PCR | |
|---|---|---|---|---|---|
| | gene array data | | | confirmation** | |
| | mean (SE) | | ratio | | ratio |
| gene¶ | genistein | control | geni/co | p value | geni/co |
| sorbitol | 5.33 (0.59) | 1.45 (0.34) | 3.68 | — | — |
| dehydrogenase | | | | | |
| prostate acid | 6.85 (0.49) | 3.38 (0.45) | 2.03 | — | — |
| phosphatase | | | | | |
| brain | 13.3 (0.34) | 6.4 (0.69) | 2.08 | 0.0003 | 2.38 |
| acid-soluble | | | | | |
| protein 1 | | | | | |
| heat shock | 8.51 (0.5) | 4.25 (0.78) | 2 | — | — |
| protein 90 | | | | | |
| MALATI* | 8.22 (0.87) | 3.88 (0.77) | 2.12 | 0.001 | 2.7 |
| heparin | 2.19 (0.21) | 6.74 (1.06) | 0.32 | 0.006 | 0.22 |
| cofactor II | | | | | |

*metastasis associated in lung adenocarcinoma transcript
**prostate tissue was re-micro dissected by LCM, RNA isolated, and used directly for qRT/PCR analysis; gene expression was normalized to that of GAPDH.
¶underlined genes have been reported to regulate cell motility

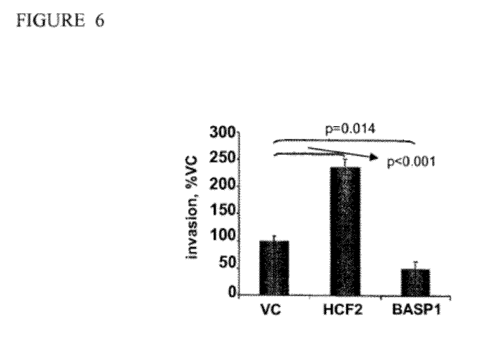

Gene array findings were first confirmed: all frozen tissues were re-cut from 24 subjects, LCM re-performed and scaled up to increase RNA yield, and qRT/PCR performed for each gene (and GAPDH for normalization) on each subject (Table 2). Functional studies were next performed, and focused upon HCF2 and BASP1. Over expression of HCF2 and BASP1 in PC3-M cells led to increased and decreased invasion, respectively, as shown in FIG. 6. Expression was confirmed by Western (not shown). It would be expected that an effective antimetastatic drug would decrease HCF2, and increase BASP1, and this is exactly what genistein does in man. Thus, this non-biased screening method selectively identified motility-associated genes provides a rigorous second independent measure of genistein's antimotility action in humans.

### Example 7

Using procedures set forth in the Detailed Description and using the appropriate starting materials, the following compounds were made or purchased commercially (compounds 8, 9, 10, and 11). Exemplary synthesis of compounds 5, 12, and 14-16 is as described herein.

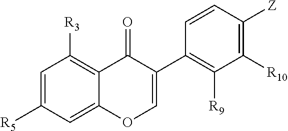

| Compound | Structure | R₅ | R₃ | Z | R₁₀ | R₉ | Dbl |
|---|---|---|---|---|---|---|---|
| 1 | 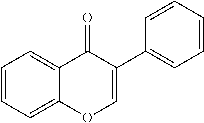 | H | H | H | H | H | + |
| 2 | 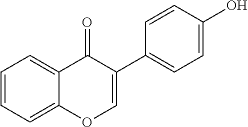 | H | H | OH | H | H | + |
| 3 | 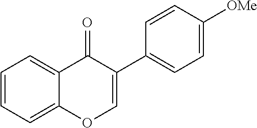 | H | H | OMe | H | H | + |
| 4 | 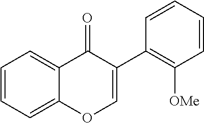 | H | H | H | H | OMe | + |
| 5 | 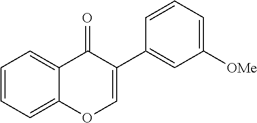 | H | H | H | OMe | H | + |
| 6 | 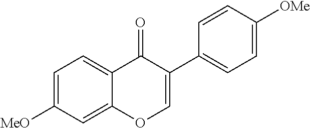 | OMe | H | OMe | H | H | + |
| 7 | 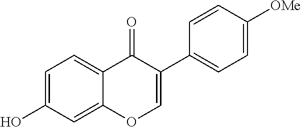 | OH | H | OMe | H | H | + |
| 8 | 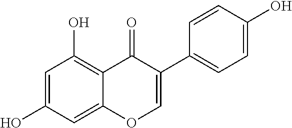 | OH | OH | OH | H | H | + |
| 9 | 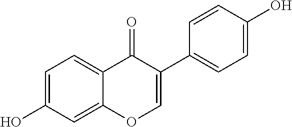 | OH | H | OH | H | H | + |
| 10 | 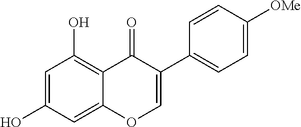 | OH | OH | OMe | H | H | + |
| 11 | 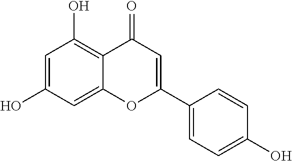 | OH | OH | OH | H | H | + |
| 12 | 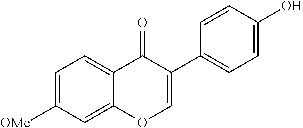 | OMe | H | OH | H | H | + |
| 13 | 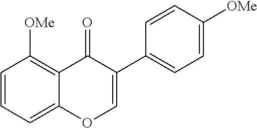 | H | OMe | OMe | H | H | + |
| 14 | 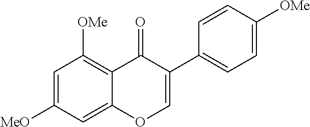 | OMe | OMe | OMe | H | H | + |
| 15 | 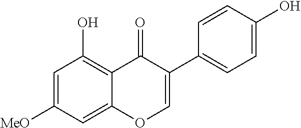 | OMe | OH | OH | H | H | + |
| 16 | 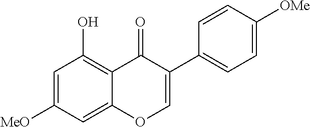 | OMe | OH | OMe | H | H | + |

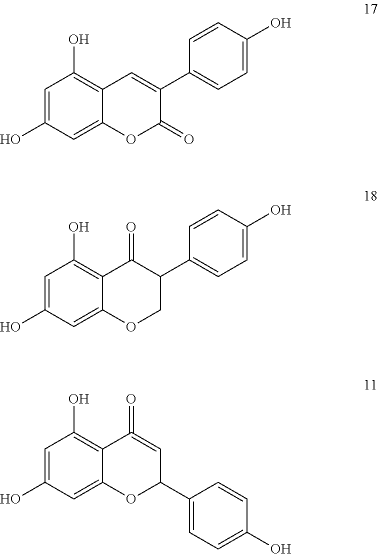

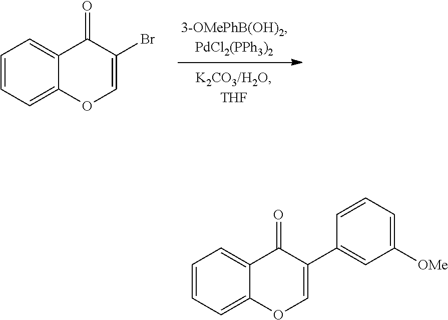

3′-methoxyisoflavone (5). Prepared using a modified procedure of Hoshino et al. (Bulletin of the Chemical Society of Japan 1988, 61, (8), 3008-3010; herein incorporated by reference in its entirety). To a 10 mL round bottom flask was added 3-bromochromone (Gammill, R. B. Synthesis-Stuttgart 1979, (11), 901-903; herein incorporated by reference in its entirety) (225 mg, 1 mmol), K2CO₃ (415 mg, 3 mmol), 3-methoxyphenylboronic acid (167 mg, 1.1 mmol), and PdCl₂(PPh₃)₂ (21 mg, 0.03 mmol). The flask was equipped with a reflux condenser and purged with N₂, followed by addition of THF/H₂O (2.5 mL/0.5 mL). The reaction was stirred at 80° C. for 4 hr. The reaction was then run through a plug of Celite and rinsed with EtOAc. The organic phase was washed with brine and dried over anhydrous Na₂SO₄. Purified by flash column chromatography (SiO₂, 15% EtOAc/Hex) and recrystallized from CH₂Cl₂/Hex to afford 5 (128 mg, 51%) as an off-white solid. Analytical data for isoflavone 5: ¹H NMR (500 MHz, CDCl₃) δ 8.34 (d, J=9.5 Hz, 1H), 8.06 (s, 1H), 7.70 (app t, J=8.5 Hz, 1H), 7.50 (d, J=8.5 Hz, 1H), 7.45 (app t, J=7.5 Hz, 1H), 7.37 (app t, J=8 Hz, 1H), 7.19 (s, 1H), 7.15 (d, J=7 Hz, 1H), 6.96 (d, J=8.5 Hz, 1H), 3.87 (s, 3H); ¹³C NMR (125 MHz, CDCl₃) δ 176.4, 159.8, 156.4, 153.4, 133.9, 133.4, 129.8, 126.7, 125.5, 124.8, 121.5, 118.3, 114.7, 114.4, 55.6; LCMS: Mass calculated for C₁₆H₁₂O₃, [M+H]⁺, 253. Found 253.

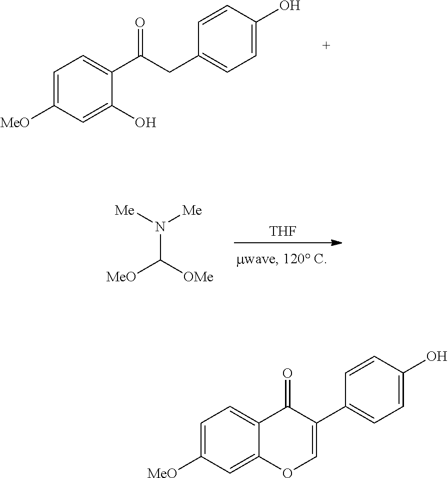

7-methoxydiadzein (12). To an oven-dried microwave vial was added 4-methoxydeoxybenzoin (73 mg, 2.83 mmol), dimethyl formamide dimethyl acetal (0.188 mL, 1.41 mmol) and THF (0.100 mL). The reaction was heated to 120° C. for 2 min. The product was recrystallized from methanol and a few drops of water to afford 12 (50 mg, 66%) as a pink powder. Analytical data for isoflavone 12: ¹H NMR (500 MHz, DMSO) δ 9.56 (s, 1H), 8.38 (s, 1H), 8.03 (d, J=9 Hz, 1H), 7.40 (d, J=9 Hz, 2H), 7.16 (s, 1H), 7.08 (d, J=9 Hz, 1H), 6.81 (d, J=9 Hz, 2H), 3.91 (s, 3H); ¹³C NMR (125 MHz, DMSO) δ 175.4, 164.3, 158.1, 157.9, 153.9, 130.8, 127.6, 124.4, 123.0, 118.3, 115.7, 115.4, 101.2, 56.8; LCMS: Mass calculated for C₁₆H₁₂O₄, [M+H]⁺, 269. Found 269.

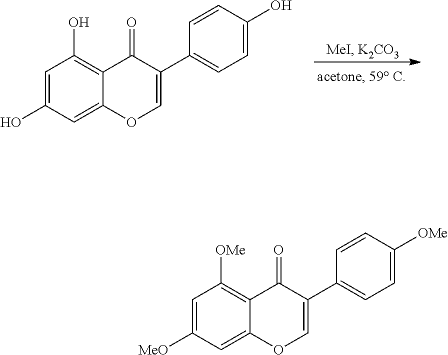

5,7,4′-trimethoxygenistein (14). To a 100 mL round bottom flask was added genistein (500 mg, 1.85 mmol) and K₂CO₃ (1.02 g, 7.4 mmol). The flask was equipped with a reflux condenser and purged with N₂. To the flask was added acetone (15 mL) and MeI (0.277 mL), and the reaction was heated to 59° C. Additional K₂CO₃ and MeI were added as needed to push the reaction. Upon completion, the reaction was allowed to cool to room temperature and was filtered to remove KI. Purified by flash column chromatography (SiO₂, 2% MeOH/CH₂Cl₂) to afford 14 (260 mg, 45%) as an off-white solid. Analytical data for isoflavone 14: ¹H NMR (500 MHz, CDCl₃) δ 7.77 (s, 1H), 7.49 (d, J=9 Hz, 2H), 6.94 (d, J=9 Hz, 2H), 6.45 (s, 1H), 6.38 (s, 1H), 3.95 (s, 3H), 3.90 (s, 3H), 3.84 (s, 3H); ¹³C NMR (125 MHz, CDCl₃) δ 175.7, 164.1, 161.7, 160.2, 159.6, 150.2, 130.6, 126.2, 124.6, 113.9, 110.2, 96.4, 92.7, 56.6, 56.0, 55.5; LCMS: Mass calculated for C₁₈H₁₆O₅, [M+H]⁺, 313. Found 313.

7-methoxygenistein (15). Prepared according to the general procedure using genistein (300 mg, 1.11 mmol), K₂CO₃ (307 mg, 2.22 mmol), acetone and MeI (0.139 mL). Purified by flash column chromatography (SiO₂, 1% MeOH/CH₂Cl₂) to afford 15 (70 mg, 22%) as an off-white solid. Analytical data for isoflavone 15: ¹H NMR (500 MHz, DMSO) δ 12.96 (s, 1H), 9.62 (s, 1H), 8.41 (s, 1H), 7.39 (d, J=8.5 Hz, 2H), 6.82 (d, J=8.5 Hz, 2H), 6.65 (s, 1H), 6.41 (s, 1H), 3.86 (s, 3H); ¹³C NMR (125 MHz, DMSO) δ 181.1, 165.9, 162.4, 158.2, 158.1, 155.1, 130.9, 123.2, 121.7, 115.8, 106.1, 98.7, 93.1, 56.8; LCMS: Mass calculated for C₁₆H₁₂O₅, [M+H]⁺, 285. Found 285.

7,4′-dimethoxygenistein (16). Prepared according to the general procedure using genistein (500 mg, 1.85 mmol), K₂CO₃ (1.02 g, 7.4 mmol), acetone (15 mL) and MeI (0.277 mL). Purified by flash column chromatography (SiO₂, 10% EtOAc/Hex) to afford 16 (270 mg, 49%) as an off-white solid. Analytical data for isoflavone 16: ¹H NMR (500 MHz, CDCl₃) δ 12.88 (s, 1H), 7.88 (s, 1H), 7.48 (d, J=8.5 Hz, 2H), 7.00 (d, J=9 Hz, 2H), 6.41 (d, J=8.5 Hz, 2H), 3.89 (s, 3H), 3.86 (s, 3H); ¹³C NMR (125 MHz, CDCl₃) δ 181.1, 165.8, 163.0, 160.0, 158.2, 152.9, 130.4, 123.9, 123.2, 114.3, 106.5, 98.4, 92.7, 56.1, 55.6; LCMS: Mass calculated for C₁₇H₁₄C₅, [M+H]⁺, 299. Found 299.

### Example 8

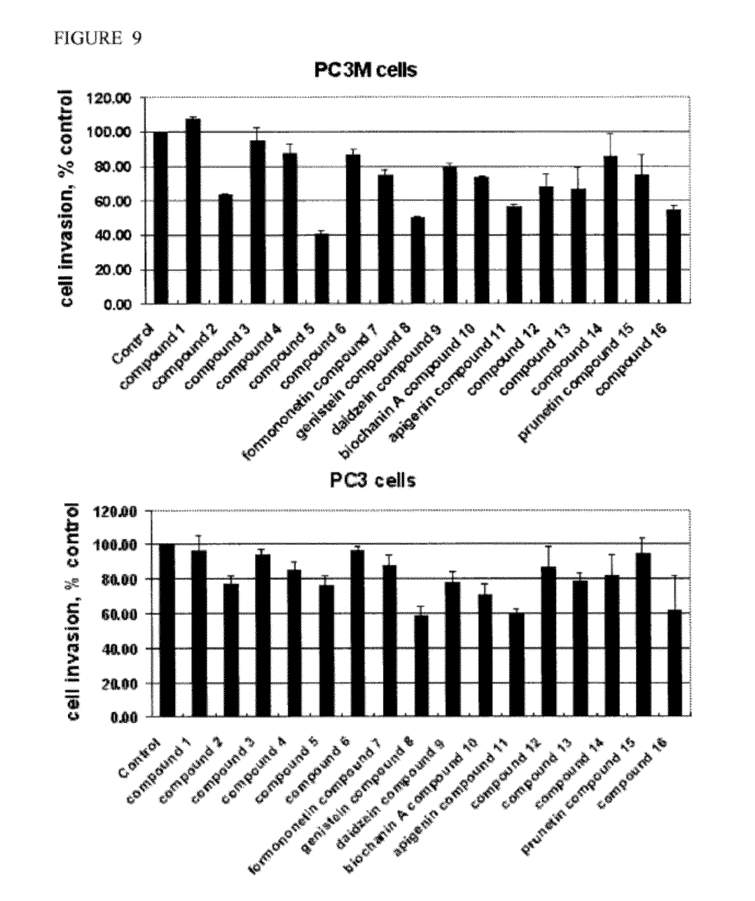

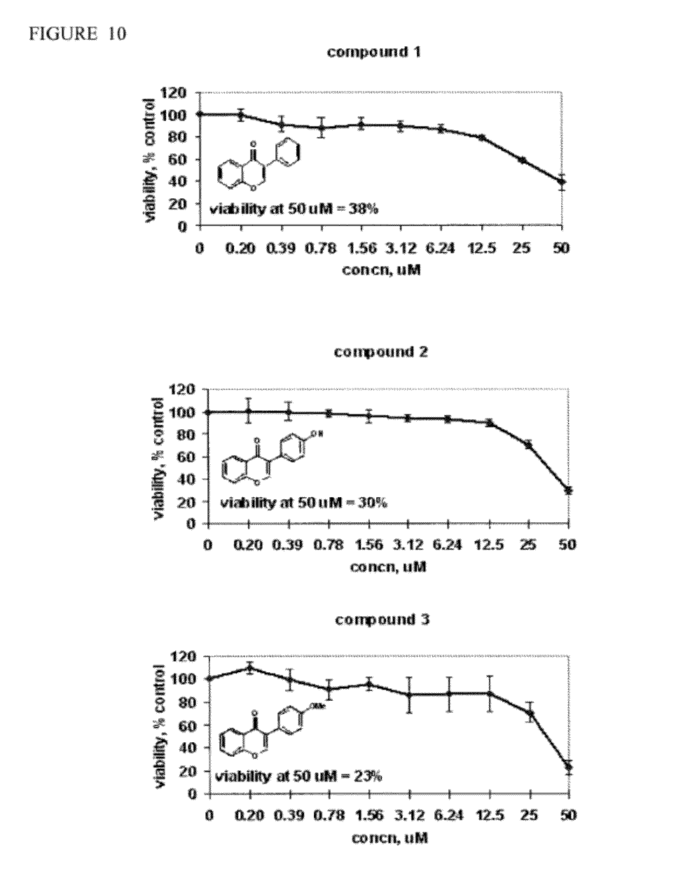

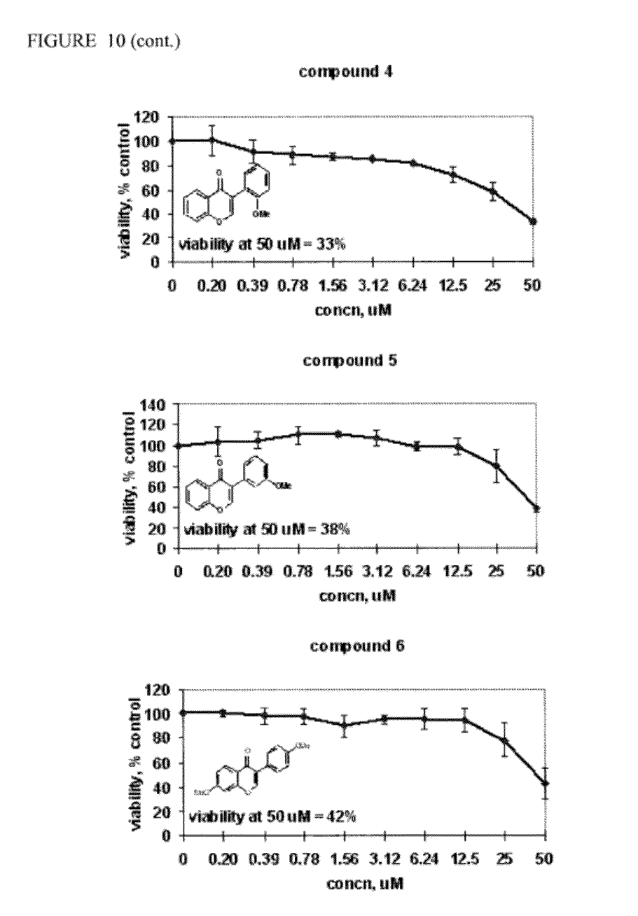

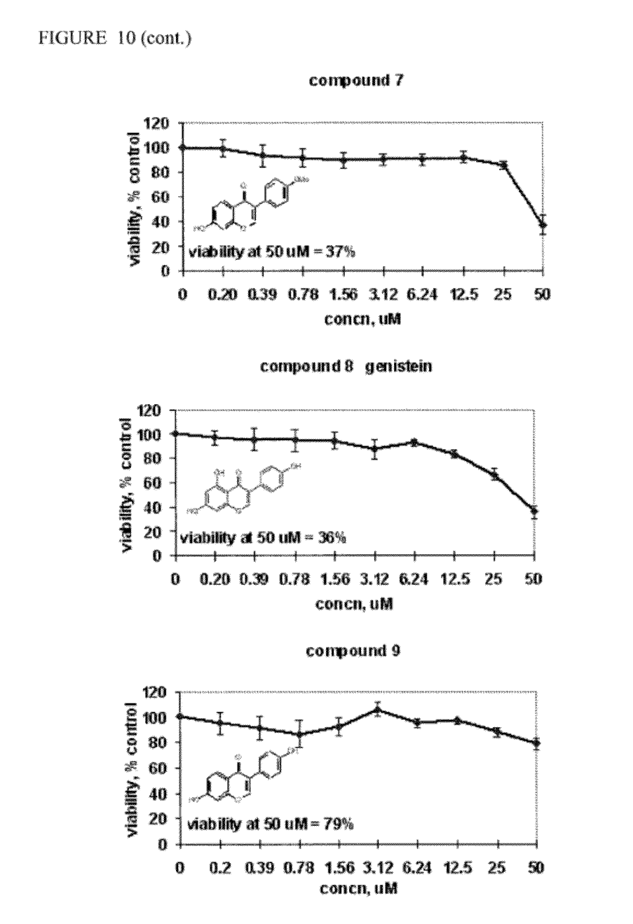

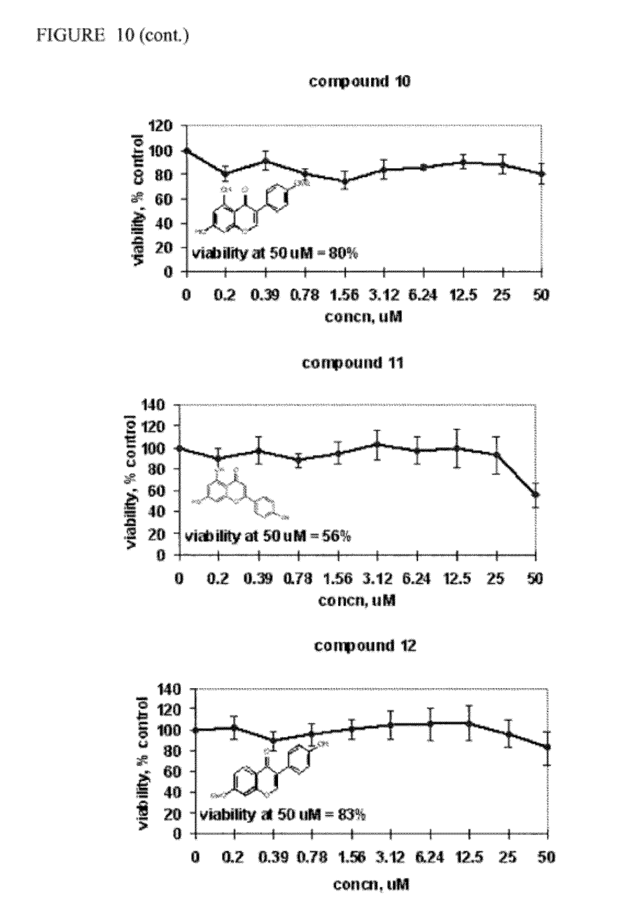

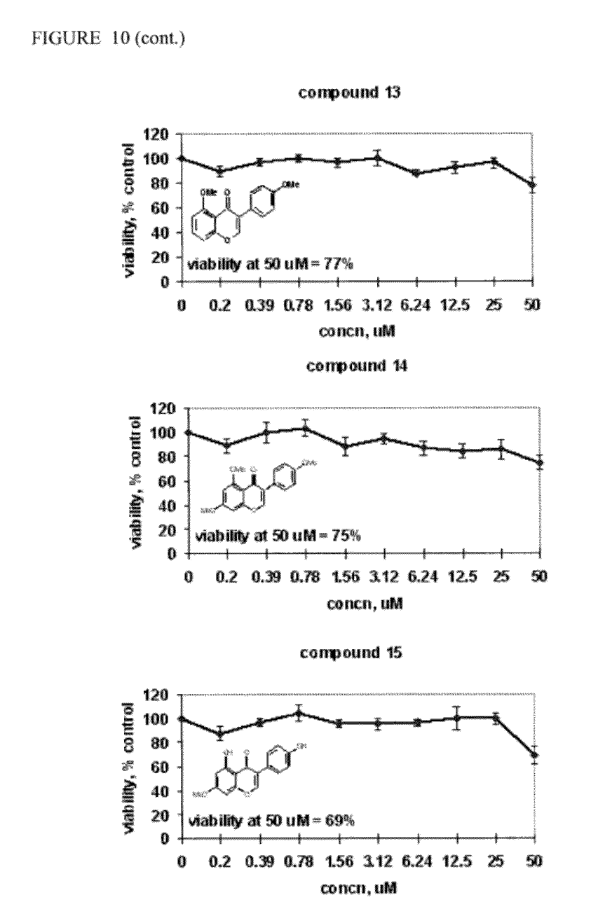

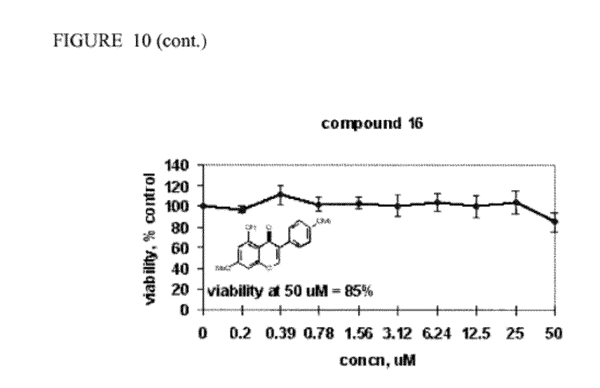

The anti-metastatic activity of the compounds of Example 7 was tested using the procedure of Example 2. Specifically, PC3-M or PC3 cells were treated with 10 μM of compound (for invasion, FIG. 9) or a range of concentrations (for growth inhibition, FIG. 10). For invasion, values are the mean±SD number of invading cells, as a percent of untreated controls, from N=3 separate assays run at different times (each assay was in replicates of N=4). Cell viability was determined by MTT assay as recited in Kyle et al., Mol. Pharmacol, 51(2):193-200 (1997); herein incorporated in its entirety. Values are the mean±SD of N=2 separate assays run at different times (N=3 for each assay), and are the percent of untreated controls.

Compounds 1, 8 and 17 did not show any significant inhibition of cell invasion whereas the remaining compounds showed varying levels of anti-metastatic activity.

1-19. (canceled)

20. A method of inhibiting MEK4 in vitro, comprising:
administering a compound having formula: 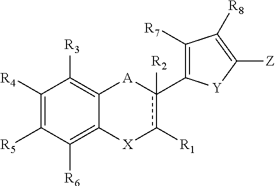
to a MEK4 enzyme in vitro; wherein A is C═O, CHOH, C═NR, or CH₂; X is O or NH; Y is O, NH, CR₉═CR₁₀, or CH═N; Z is OH, OCH₃, halogen, or H provided that one of R₇ or R₈ is OH or OCH₃; the dashed line represents an optional double bond; R is H or a substituted or unsubstituted alkyl group; R₁ is selected from the group consisting of H and substituted or unsubstituted alkyl groups; R₂ is selected from the group consisting of H, OH, F and Cl; or is absent when the optional double bond is present; R₃, R₄, R₅, R₆, R₇, R₈, R₉ and R₁₀ are each independently selected from the group consisting of OH, F, Cl, Br, I, CN, NO₂, COOR, CONH₂, and substituted and unsubstituted alkyl and alkoxy groups.

21. The method of claim 20, further comprising the step of detecting MEK4 enzyme activity.

22. The method of claim 21, wherein said MEK4 enzyme activity comprises detecting activity of a MEK4 enzyme pathway member.

23. The method of claim 22, wherein said MEK4 enzyme pathway member is selected from the group consisting of: p38 MAPK, MAPK APK2, HSP 27, or MMP-2.

24.-28. (canceled)

29. A pharmaceutical preparation comprising a compound having formula: 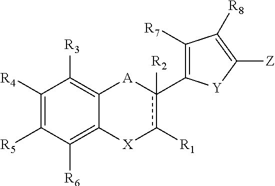
wherein A is C═O, CHOH, C═NR, or CH₂; X is O or NH; Y is 0, NH, CR₉═CR₁₀, or CH═N; Z is OH, OCH₃, halogen, or H provided that one of R₇ or R₈ is OH or OCH₃; the dashed line represents an optional double bond; R is H or a substituted or unsubstituted alkyl group; R₁ is selected from the group consisting of H and substituted or unsubstituted alkyl groups; R₂ is selected from the group consisting of H, OH, F and Cl; or is absent when the optional double bond is present; R₃, R₄, R₅, R₆, R₇, R₈, R₉ and R₁₀ are each independently selected from the group consisting of OH, F, Cl, Br, I, CN, NO₂, COOR, CONH₂, and substituted and unsubstituted alkyl and alkoxy groups; wherein said compound is not genistein.

30. The composition of claim 29 wherein, if Z is H, one of R₇ or R₈ is OH or OCH₃.

31. The composition of claim 29, wherein R₃, R₄, R₅, and R₆ are each H.

32. The composition of claim 29, wherein Z is OCH₃, halogen, or H.

33. The pharmaceutical composition of claim 29, wherein said compound is selected from the group consisting of

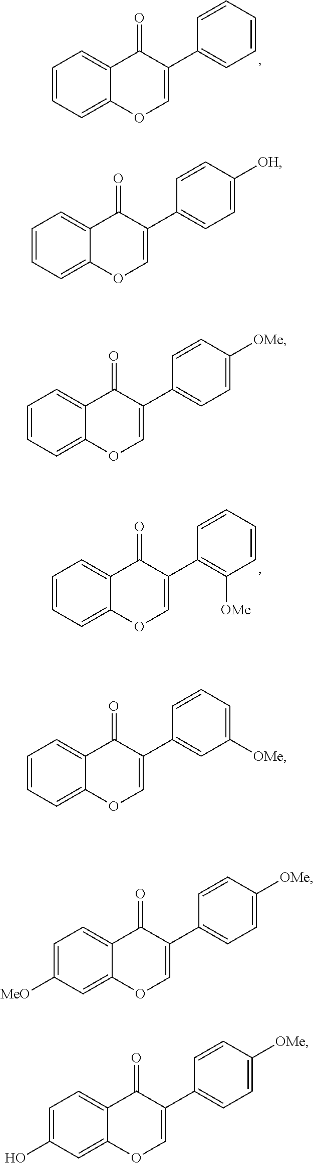

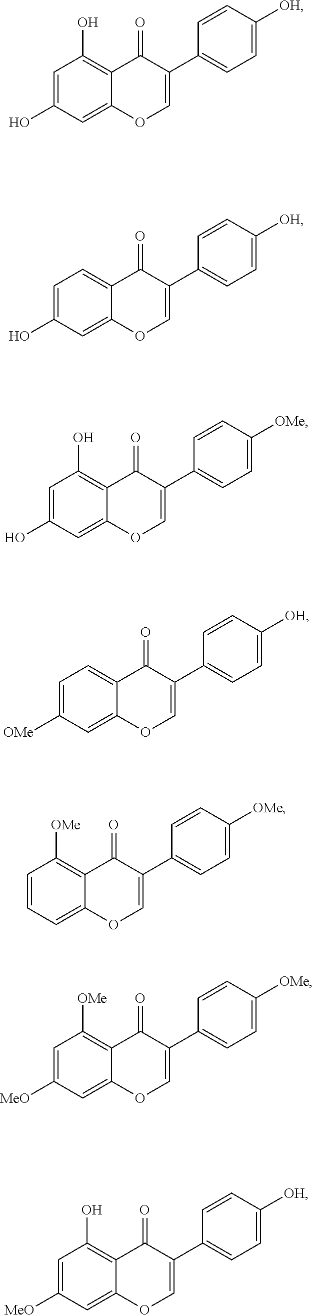

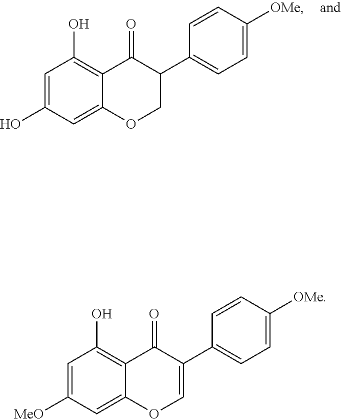

34. A composition comprising a compound selected from the group consisting of 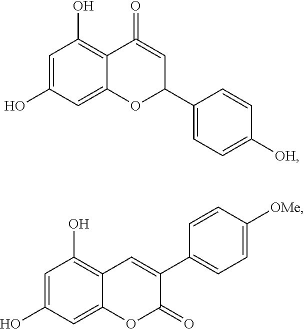
and a compound described by the following formula: 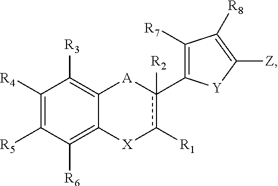
including salts, esters, and prodrugs thereof;
including both R and S enantiomeric forms and racemic mixtures thereof;
wherein A is selected from the group consisting of O, C═O, CHOH, C═NR, and CH2;
wherein X is selected from the group consisting of C═O, O, and NH;
wherein Y is selected from the group consisting of O, NH, CR₉═CR₁₀, and CH═N;
wherein Z is selected from the group consisting of OH, OCH₃, halogen, and H provided that one of R₇ or R₈ is OH or OCH₃;
wherein R is selected from the group consisting of H and a substituted or unsubstituted alkyl group;
wherein R₁ is selected from the group consisting of H, C═O, and substituted or unsubstituted alkyl groups;
wherein R₂ is absent or is selected from the group consisting of H, OH, F and Cl;
wherein R₃, R₄, R₅, R₆, R₇, R_{g}, R₉ and R₁₀ are each independently selected from the group consisting of OH, F, Cl, Br, I, CN, NO₂, COOR, CONH₂, and substituted and unsubstituted alkyl and alkoxy groups; and
wherein R is, at each occurrence, independently selected from the group consisting of hydrogen, a substituted or unsubstituted alkyl group, and a substituted or unsubstituted alkoxy group.

35. The composition of claim 33, wherein said compound is selected from the group consisting of 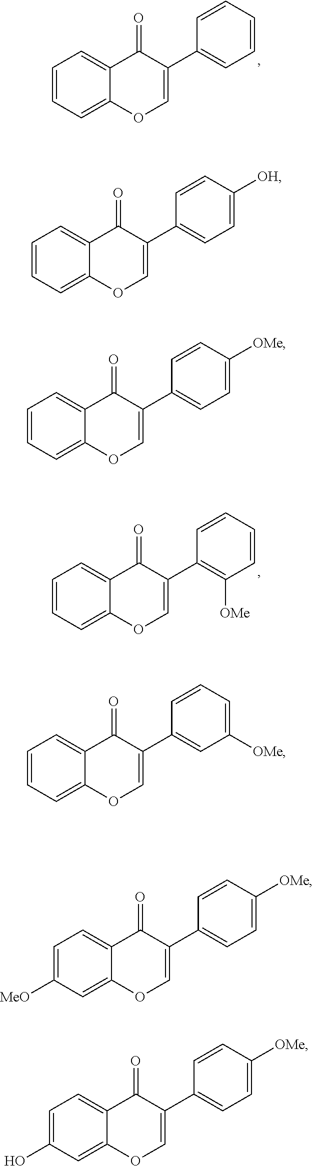 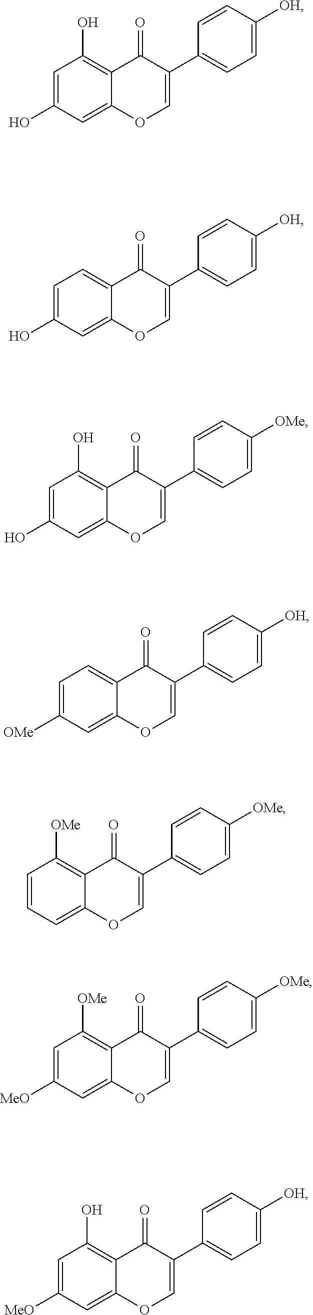 [chemical structure]


SEQUENCE LISTING (9 sequences)

SEQ ID NO: 1 - DNA, 20 bp, Artificial Sequence
  Synthetic
ttcctcctttgtctcccagc

SEQ ID NO: 2 - DNA, 20 bp, Artificial Sequence
  Synthetic
attcctcctttgtctcccag

SEQ ID NO: 3 - DNA, 19 bp, Artificial Sequence
  Synthetic
attcctcctttgtctccca

SEQ ID NO: 4 - DNA, 21 bp, Artificial Sequence
  Synthetic
gcctctttatcacctaccaca

SEQ ID NO: 5 - DNA, 20 bp, Artificial Sequence
  Synthetic
aauucctcctttgtcuccca

SEQ ID NO: 6 - DNA, 20 bp, Artificial Sequence
  Synthetic
gucuctctatgtgtggguuu

SEQ ID NO: 7 - DNA, 20 bp, Artificial Sequence
  Synthetic
ugugugttctcagtcucucu

SEQ ID NO: 8 - DNA, 20 bp, Artificial Sequence
  Synthetic
cuccucgtccaatttcucca

SEQ ID NO: 9 - DNA, 20 bp, Artificial Sequence
  Synthetic
ggcuugctgtggtcgaaggc